(19) United States
(12) Patent Application Publication
(10) Pub. No.: US 2013/0288389 A1
(43) Pub. Date: Oct. 31, 2013

(54) METHODS FOR AMPLIFICATION AND DETECTION OF PRIONS

(71) Applicants: Christina D. Orru, Hamilton, MT (US); Byron W. Caughey, Hamilton, MT (US); Franziska Kuhn, Schlieren-Zurich (CH); Bjorn Schroder, Schlieren-Zurich (CH); Alex Raeber, Schlieren-Zurich (CH)

(72) Inventors: Christina D. Orru, Hamilton, MT (US); Byron W. Caughey, Hamilton, MT (US); Franziska Kuhn, Schlieren-Zurich (CH); Bjorn Schroder, Schlieren-Zurich (CH); Alex Raeber, Schlieren-Zurich (CH)

(73) Assignees: Prionics AG, Schlieren-Zurich (CH); THE UNITED STATES OF AMERICA, as represented by the Secretary, Department of Health and Human, Bethesda, MD (US)

(21) Appl. No.: 13/979,843

(22) PCT Filed: Jan. 17, 2012
(86) PCT No.: PCT/US12/21561

§ 371 Date: Jul. 15, 2013

Related U.S. Application Data
(60) Provisional application No. 61/433,881, filed on Jan. 18, 2011.

(51) Int. Cl. G01N 33/68

(52) U.S. Cl. CPC G01N 33/6896
USPC 436/501

(57) ABSTRACT

Methods are disclosed for detecting prions and/or prion disease-associated forms of prion protein. These methods provide sensitive and specific identification of prions in both biological and environmental samples. These methods include the use of both immunoprecipitation and an amplification assay that uses shaking in the absence of sonication, such as QuIC(SQ) or RT-QuIC(RTQ). In specific non-limiting examples, the methods include the use of monoclonal antibody 15B3 and/or RT-QuIC(RTQ), and/or a substrate replacement step.

### PRIORITY CLAIM

This claims the benefit of U.S. Patent Application No. 61/433,881, filed Jan. 18, 2011, which is incorporated by reference herein in its entirety.

### PARTIES TO JOINT RESEARCH AGREEMENT

The Government of the United States of America, U.S. Department of Health and Human Services, as represented by the National Institute of Allergy and Infectious Disease, an institute of the National Institutes of Health; and Prionics AG are parties to a joint research agreement related to the technology disclosed herein.

### FIELD OF THE DISCLOSURE

The present disclosure relates to methods and compositions for the detection of infectious proteins or prions in samples, including the diagnosis of prion related diseases.

### BACKGROUND

The transmissible spongiform encephalopathies (TSEs) or prion diseases are fatal neurodegenerative disorders that include human Creutzfeldt-Jakob disease (CJD), bovine spongiform encephalopathy (BSE), sheep scrapie, cervid chronic wasting disease (CWD), and transmissible mink encephalopathy (TME). The infectious agent, or prion, of the TSEs appears to be composed primarily of an abnormal, misfolded, oligomeric, and usually partially protease-resistant form of prion protein (e.g., PrP-res, PrP^{vCJD}, PrP^{Sc}). PrP-res is formed post-translationally from the normal cellular prion protein (PrP^{C}) (Borchelt et al., J Cell Biol, 110, 743-752, 1990; Caughey and Raymond, J Biol Chem, 266, 18217-18223, 1991). PrP-res, which in purified form can resemble amyloid fibrils, induces the polymerization and conformational conversion of PrP^{C} to infectious PrP-res/PrP^{Sc} (Castilla et al., Cell, 121, 195-206, 2005; Deleault et al., Proc Natl Acad Sci USA, 104, 9741-9746, 2007) or to PrP^{Sc}-like partially protease-resistant forms in a variety of in vitro reactions (Caughey et al., Annu Rev Biochem, 78, 177-204, 2009; Deleault et al., 2007, supra; Kocisko et al., Nature, 370, 471-474, 1994; Saborio et al., Nature, 411, 810-813, 2001). These studies demonstrate that PrP-res can self-propagate, and although the mechanism is not fully understood, it appears to be a seeded or templated polymerization (Gadjusek, Infectious amyloids: Subacute Spongiform Encephalopathies as Transmissible Cerebral Amyloidoses. In Fields, B. N., Knipe, D. M., and Howley, P. M. (Eds.), Field's Virology, Lippincott-Raven, Philadelphia, pp. 2851-2900, 1996; Horiuchi et al., Proc Natl Acad Sci USA, 97, 5836-5841, 2000; Jarrett and Lansbury, Jr., Cell, 73, 1055-1058, 1993).

The ability to detect prions rapidly and sensitively would be an important asset in managing TSEs. Early prion detection in individuals is critical to the prevention of spread and the initiation of potential treatments. Prions can be found in a wide variety of tissues and accessible bodily fluids from infected mammalian hosts, including blood (Brown et al., Transfusion, 38, 810-816, 1998; Manuelidis et al., Science, 200, 1069-1071, 1978; Mathiason et al., Science, 314, 133-136, 2006; Saa et al., 2006a; Terry et al., J Virol, 83, 12552-12558, 2009; Thorne and Terry, J Gen Virol, 89, 3177-3184, 2008), breast milk (Konold et al., BMC Vet Res, 4, 14, 2008; Lacroux et al., PLoS Pathog, 4, e1000238, 2008), saliva (Mathiason et al., Science, 314, 133-136, 2006; Vascellari et al., J Virol, 81, 4872-4876, 2007), urine (Gregori et al., Emerg Infect Dis, 14, 1406-1412, 2008; Murayama et al., PLoS ONE, 5, 2007), feces (Safar et al., J Infect Dis, 198, 81-89, 2008), and nasal fluids (Bessen et al., PLoS Pathogens, 6, e1000837, 2010). In most cases, the ability to rapidly measure prion infectivity in these fluids is limited by the low amount of infectious agent. Knowledge of the prion titers in these fluids or tissues and their products is important for prion diagnosis and in assessing the public health exposure risks to those materials. Furthermore, is useful to be able to detect prions in environmental samples, food products and animal feed. Thus, a need remains for rapid, sensitive and specific assays for prions.

### SUMMARY OF THE DISCLOSURE

Methods are disclosed for detecting prion proteins. These methods provide sensitive and specific identification of prions in both biological and environmental samples. These methods include the use of both immunoprecipitation and an amplification assay that uses shaking in the absence of sonication, such as QuIC or RT-QuIC.

In some embodiments, methods are provided for detecting prion protein, that include contacting a sample with an effective amount of an antibody that specifically binds a PrP-res for sufficient time to form an immune complex, and mixing the immune complex with purified recombinant prion protein (rPrP^{C}) to make a reaction mixture. The immune complex can be separated from the sample. An amplification reaction is performed, that includes incubating the reaction mixture to permit coaggregation of the PrP-res with the rPrP^{C} in the reaction mixture and maintaining incubation conditions that promote coaggregation of the rPrP^{C} with the PrP-res to result in a conversion of the rPrP^{C} to rPrP-res^{(Sc)} while inhibiting (e.g., preventing) development of spontaneously formed rPrP-res^{(spon)}. The reaction mixture is agitated, wherein agitating comprises shaking the reaction mixture without sonication. rPrP-res^{(Sc)} is detected in the reaction mixture, wherein detection of rPrP-res^{(Sc)} in the reaction mixture indicates that PrP-res is present in the sample.

In some embodiments, amounts of rPrP-res^{(Sc)} in the reaction mixture can be quantitated. In additional embodiments, detecting rPrP-res^{(Sc)} in the reaction mixture includes the use of Thioflavin T (ThT).

In further embodiments, the rPrP^{C} can be replenished by adding additional rPrP^{C} substrate prior to detecting in the reaction mixture.

In additional embodiments, the immune complex can be pre-incubated, such as with a buffer comprising a detergent, such as sodium dodecyl sulfate, prior to performing the amplification reaction. The antibody that specifically binds PrP-res can be bound to a solid substrate, including but not limited to, magnetic beads. In further embodiments, the antibody is 15B3.

In additional embodiments, the rPrP^{C} can be a chimeric rPrP^{C}, such as a chimeric hamster-sheep rPrP^{C}. In specific non-limiting examples, the assay can detect vCJD and other forms of STEs.

In one specific, non-limiting example, methods for detecting prion protein in a biological sample are provided, wherein the methods include contacting the biological sample, such as plasma, blood, serum, cerebral spinal fluid or a tissue sample with an effective amount of antibody 15B3 coupled to a solid substrate for sufficient time to form an immune complex on the solid substrate. The immune complex on the substrate is separated from the other components of the biological sample. The immune complex on the solid substrate is incubated with a buffer comprising about 0.01% to about 0.05% sodium dodecyl sulfate. The immune complex on the solid substrate is mixed with purified recombinant prion protein (rPrP^{C}), such as hamster sheep chimeric recombinant prion protein (rPrP^{C}), and Thioflavin T, to make a reaction mixture and an amplification reaction is performed. The amplification reaction includes: (a) incubating the reaction mixture to permit coaggregation of the PrP-res with the rPrP^{C} that are present in the reaction mixture; (b) maintaining incubation conditions that promote coaggregation of the rPrP^{C} with the PrP-res to result in a conversion of the rPrP^{C} to rPrP-res^{(Sc)} while inhibiting development of rPrP-res^{(spon)}; (c) agitating aggregates formed during step (i), wherein the reaction mixture is shaken and then not shaken for a substantially equal period of time, such as shaken for about 60 seconds and then not shaken for about 60 seconds, or shaken for about 30 seconds and then not shaken for about 30 seconds; (d) adding additional recombinant prion protein (rPrP^{C}), such as hamster sheep chimeric prion protein, to the reaction mixture prior to the formation of detectable rPrP-res^{(Sc)}. The steps, such as steps (c) and (d) can optionally be repeated. In some embodiments, the rPrP-res^{(Sc)} in the reaction mixture is detected using ThT fluorescence, wherein fluorescence of the reaction mixture indicates that PrP-res was present in the sample. In additional examples, the rPrP^{C} can be replenished by adding additional rPrP^{C} substrate prior to detecting rPrP-res^{(Sc)} in the reaction mixture. In additional examples,

The foregoing and other features and advantages of the invention will become more apparent from the following detailed description of a several embodiments which proceeds with reference to the accompanying figures.

### BRIEF DESCRIPTION OF THE FIGURES

FIGS. 1a-1b. IP-S-QuIC detection of ≧10 fg human vCJD PrP-res spiked into human plasma. Dilutions of human non-prion (tumor, T) control or vCJD brain homogenates were spiked into 500 μl of human plasma to give final dilutions of 4×10⁻⁷ (T); and 4×10⁻⁷, 4×10⁻⁹, and 4×10⁻¹⁰ (vCJD; containing ˜10 pg, 100 fg and 10 fg PrP-res, respectively). PrP^{vCJD} was immunoprecipitated and subjected to S-QuIC as described in Materials and Methods. The first S-QuIC round was at 50° C. for 8 hour (h) (FIG. 1a) and 1/10 of the first-round reaction volume was used to seed the 2^{nd} round (45° C. for 10 h) (FIG. 1b). Plasma-free positive and negative control reactions were seeded directly with 2 μl of 5×10⁻⁷ dilutions of hamster uninfected (N) or scrapie (Sc) brain, the latter containing ˜100 fg PrP^{res} seed. Hamster rPrP^{C} 23-231 was used as a substrate in all reactions and comigrated with the 25 kDa marker. PK-digested products were analyzed by immunoblot using the polyclonal R20 antibody as previously reported (24). Open circles mark 17-kDa fragments and brackets indicate the lower molecular weight bands (10-13 kDa).

FIGS. 2a-2b. IP-S-QuIC detection of endogenous PrP^{sc} in plasma of scrapie-infected hamsters by IP-S-QuIC. (FIG. 2a) Plasma samples from scrapie 263K and uninfected (N) hamsters (500 μl) were subjected to IP-S-QuIC as described in the Examples Section with the 1^{st} round S-QuIC at 50° C. for 10 hours (h) and the 2^{nd} round (FIG. 2b) at 50° C. for 8 h, except for lanes marked with asterisks which show the 1^{st} round products seeded with sample #6 for comparison. Plasma-free positive and negative control reactions, rPrP^{C} 23-231 substrate and analysis of PK-digested products were as described for FIG. 1. Open circles mark 17-kDa fragments and brackets indicate the lower molecular weight bands (10-13 kDa).

FIGS. 3a-3b. IP-RT-QuIC detection of endogenous PrP^{Sc} in plasma and serum of scrapie-infected hamsters. (FIG. 3a) IP-RT-QuIC analyses of plasma samples from a scrapie 263K and a normal hamster, and a serum sample from a scrapie 263K hamster. (FIG. 3b) Analyses of plasma samples from nine scrapie 263K and one uninfected hamster. In all cases, 500 μl samples were immunoprecipitated using 15B3-coated beads for ˜20 h at 37° C. One fifth of the beads was pre-incubated with 0.05% SDS in PBS at room temperature for ˜20 minutes and used to seed RT-QuIC containing 300 mM NaCl. RT-QuIC reactions were incubated at 42° C. and hamster rPrP^{C} 90-231 was used as a substrate in all reactions. The vertical axes indicate the average fluorescence from 4 replicate reaction wells. Error bars show standard deviations for selected sets of replicates in (FIG. 3a). In (FIG. 3b), all individual reactions that registered positive fluorescence achieved nearly identical maximal fluorescence values (˜260 k units), but in many of the scrapie-seeded cases, only a subset of replicate reactions rose above background fluorescence within 63 h. With such all-or-nothing responses among replicates, standard deviations cannot be calculated from all of the replicates; instead, on the right, the fraction of positive wells per total replicates is indicated at the end of the reactions. Error bars representing standard deviations calculated for the positive replicates (only) at >40-h time points barely, if at all, exceeded the size of the symbols, and therefore are not shown.

FIGS. 4a-4b. eQuIC detection of human PrP^{cCJD} spiked into human plasma. Dilutions of human non-prion (tumor and Alzheimer's disease) control or vCJD brain tissues were spiked into 500 μl of human plasma to give final dilutions of 4×10⁻⁷ (tumor and Alzheimer's disease); and 4×10⁻¹², 4×10⁻¹³ and 4×10⁻¹⁴ (vCJD; containing ˜100 ag, 10 ag and 1 ag PrP-res, respectively). PrP^{vCJD} was immunoprecipitated using 15B3-coated beads (FIG. 4a) or mock anti-IgM-coated beads (FIG. 4b) and a portion of the beads were used to seed replicate eQuIC reactions. After 24 h the substrate was replaced. The chimeric Ha-S rPrP^{C} was used as a substrate in all reactions. The vertical axes indicate the average fluorescence from 4 replicate wells and the fractions on the right indicate the positive/total replicate reactions associated with the adjacent traces.

FIGS. 5a-5b. eQuIC detection of endogenous PrP^{Sc} in plasma of scrapie-infected hamsters. (FIG. 5a). eQuIC analysis of plasma samples (without preclearing) from 8 uninfected hamsters and 6 scrapie-infected hamsters, with one collected at 30 dpi (preclinical) and 5 at 80 dpi (near-terminal). The vertical axis indicates the average fluorescence from 4 replicate wells and the fractions on the right indicate the positive/total replicate reactions associated with the adjacent traces. Although all replicate reactions seeded with the scrapie samples were positive, submaximal average fluorescence observed for 3 of the samples at 60 h. In the latter cases, the bead distribution in the well partially interfered with fluorescence readings; when such wells (n=3) were reread at 64 h after manual stirring with a pipette, the fluorescence achieved maximal levels (grey trace). In contrast, stirring uninfected control wells (n=4) did not increase their fluorescence. Hamster rPrP^{C} 90-231 was used as a substrate. (FIG. 5b). eQuIC analysis of precleared plasma samples from 3 uninfected and 7 scrapie-infected hamsters (3 collected at 80 dpi; 2 at 30 dpi; 1 at 10 dpi).

FIG. 6. Schematic diagram of potential mechanisms of substrate replacement effect.

FIGS. 7a-7b. Better IP-S-QuIC sensitivity and consistency of PrP^{Sc} detection in spiked human plasma using 15B3 vs. mock beads. (FIG. 7a) Comparison of 15B3 vs mock beads with 2-h IP from 100 μl plasma and 2-round S-QuIC. Dilutions of hamster uninfected “normal” (N) or scrapie 263K (Sc) brain homogenates were spiked into 100 μl of human plasma to give final brain dilutions of 10⁻⁸ (N); and 10⁻⁸, 10⁻⁹ and 10⁻¹⁰ (Sc; ˜100, 10, and 1 fg PrP-res, respectively). PrP^{Sc} was immunoprecipitated using 40 μl (1.6×10⁷ total beads) of 15B3-coated beads (15B3) or mock anti-IgM-coated beads (C) for 2 h at 37° C. Beads were resuspended in 10 μl of PBS. One fifth of the beads was used to seed a 1^{st} round 5-QuIC at 50° C. for 10 h and 1/10 of the 1^{st} round reaction volume was used to seed the 2^{nd} round (50° C. for 10 h). (FIG. 7b) 15B3 beads with 20-h IP from 500 μl plasma and single-round S-QuIC. Dilutions of N or Sc brain homogenates were spiked into 500 μl of human plasma to give final brain tissue dilutions of 2×10⁻⁸ (N); and 2×10⁻⁸-2×10⁻¹¹ (Sc; containing ˜1 pg-1 fg PrP-res, respectively). PrP^{Sc} was immunoprecipitated using 15B3 beads for ˜20 h at 37° C. The remainder of the protocol was as in (FIG. 7a) except that only a single-round S-QuIC at 50° C. for 10 h was performed. Plasma-free positive and negative control reactions were seeded directly with 2 μl of 5×10⁻⁷ dilutions of hamster N or Sc brain, the latter containing ˜100 fg PrP-res seed. Hamster rPrP^{C} 23-231 was used as a substrate in all S-QuIC reactions. PK-digested products were analyzed by immunoblot using the polyclonal R20 antibody as previously reported (Orru et al., 2009. Protein Eng Des Sel 22:515-521, 2009). Open circles mark 17-kDa fragments and brackets indicate the lower molecular weight bands (10-13 kDa).

FIGS. 8a-8b. SDS pre-treatment of 15B3-bound PrP^{Sc} accelerates RT-QuIC detection. Dilutions of hamster N or Sc 263K brain homogenates were spiked into 500 μl of human plasma to give final brain dilutions of 2×10⁻⁷ (containing ˜10 pg PrP-res in the case of Sc). IP incubations with beads were for ˜20 h at 37° C. One fifth of the beads was used to seed RT-QuIC (FIG. 8a) and an equivalent number of beads was pre-incubated with 0.05% SDS in PBS at room temperature for ˜20 minutes and used to seed RT-QuIC reactions containing 300 mM NaCl (FIG. 8b). Reactions were incubated at 42° C. and hamster rPrP^{C} 90-231 was used as a substrate in all reactions. The vertical axis indicates the average fluorescence from 4 replicate wells and the fractions on the right indicate the positive/total replicate reactions associated with the adjacent traces.

FIGS. 9a-9c. Improved RT-QuIC detection of 15B3-bound human PrP^{vCJD} with hamster-sheep chimeric rPrP^{C} (Ha-S rPrP^{C}) vs. human rPrP^{C} 23-231 with NaCl variation. Dilutions of human non-prion (tumor) control or vCJD brain tissues were spiked into 500 μl of human plasma to give final dilutions of 4×10⁻⁷ (FIGS. 9a-9b), containing ˜10 pg PrP-res, in the case of vCJD) or 4×10⁻⁷ and 4×10⁻⁹ and 4×10⁴⁰ (FIG. 9c, containing ˜10 pg, 100 fg and 10 fg PrPr^{res}, respectively in the case of vCJD). The samples were subjected to IP-RT-QuIC as described in the Materials and Methods section except for indicated variations in NaCl concentration and rPrP^{C} substrate. The vertical axis indicates the average fluorescence from 4 replicate wells and the fractions on the right indicate the positive/total replicate reactions associated with the adjacent traces.

FIGS. 10a-10b. Comparison of 15B3 beads to Magnabeads in eQuIC. Dilutions of human non-TSE Alzheimer's disease (AD) control or vCJD brain tissues were spiked into 0.5 ml of human plasma to give final dilutions of 4×10⁻⁷ (AD); and 4×10⁻⁷, 4×10⁻¹⁰, 4×10⁻¹³ and 4×10⁻¹⁴ (vCJD; containing ˜10 pg, 10 fg, 10 ag and 1 ag PrP-res, respectively). PrP^{vCJD} was immunoprecipitated using 1.6×10⁷ total 15B3-coated beads (FIG. 10a) or an equivalent number of MAGNABIND™ beads (FIG. 10b) for ˜20 h at 37° C. using immunoprecipitation buffer (Prionics). Beads were washed twice with wash buffer (Prionics) and resuspended in 10 μl of PBS. The remainder of the protocol was as described in Material and Methods, starting with the preincubation in 0.05% SDS in PBS. The vertical axis indicates the average fluorescence from four replicate wells and the fractions on the right indicate the positive/total replicate reactions associated with the adjacent traces. Similar results were obtained using the MAGNABIND™ PrP^{Sc} capture conditions (Miller and Supattapone, 2011, J. Virol. 85:2813-2817).

FIG. 11. Improved speed & sensitivity of IP-RTQ using higher 15B3 content on beads. Dilutions of hamster normal (NBH) or scrapie 263K brain homogenates were spiked into 500 μl of human plasma to give final brain tissue dilutions of 2×10⁻⁹ (NBH); and 2×10⁻⁹ and 2×10⁻¹⁰ (263K; containing ˜100 or 10 fg PrP-res, respectively). PrP-res was immunoprecipitated using 40 μl of 15B3 beads for ˜20 hours at 37° C. Beads were washed twice with 0.2% Sarkosyl/TBS and resuspended in 10 μl of PBS. Following a 0.05% SDS pre-treatment, one fifth of the beads was used to seed RTQ reactions. Hamster rPrP^{C} 90-231 was used as a substrate in all reactions. The vertical axis indicates the average fluorescence from 2 replicate wells.

FIG. 12. 15B3 antibody titration for eQuIC detection of sheep scrapie brain homogenate spiked into plasma. The results demonstrate that increasing the amount of 15B3 results in improved sensitivity of the sheep eQuIC, which allows faster detection of Sheep scrapie brain tissue dilutions containing ˜100 fg of PrP-res in 0.5 ml of plasma.

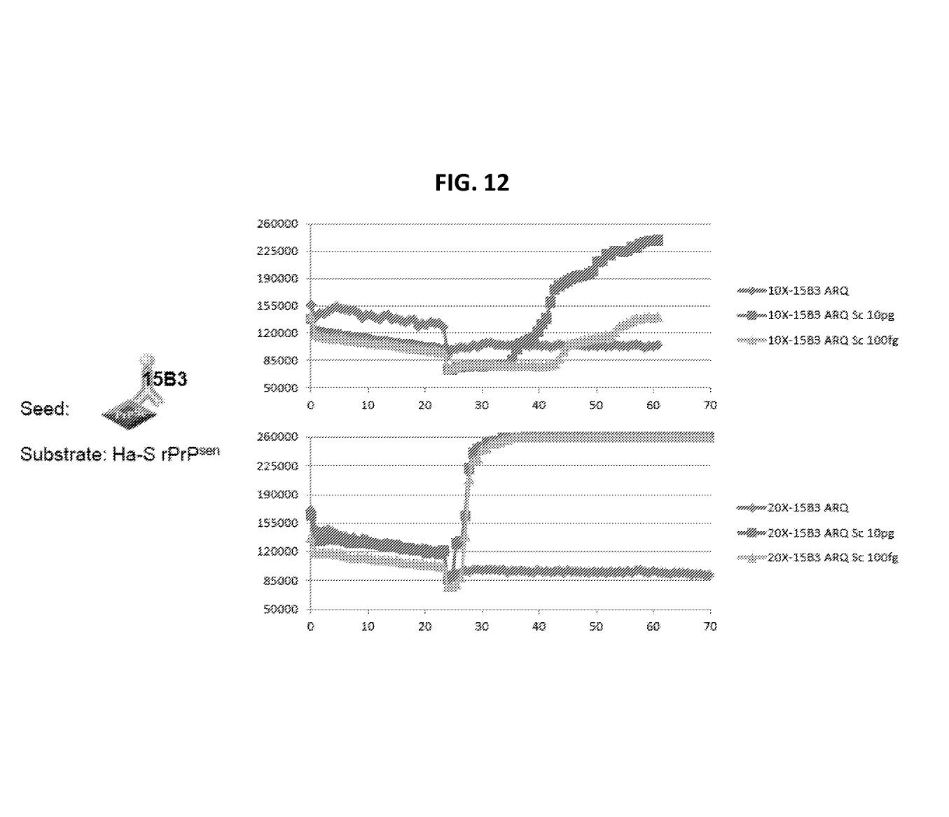

FIG. 13. eQuIC detection of ARQ sheep brain homogenate in spiked sheep plasma. The assay provided detection of ≧−100 ag PrP-res (5×10⁻¹³ dilution of brain tissue) in 500 ul of sheep plasma.

FIG. 14. eQuIC detection of endogenous PrP-res in plasma of scrapie positive sheep. The assay detected endogenous PrP-res in plasma from three slinically affected scrapie-infected sheep. No prions were detected in plasma samples from four non-infected sheep.

FIG. 15. Sensitivity of detection of sCJD brain homogenate spiked into plasma by e-QuIC. The assay detected sCJD brain homogenate spikes containing as little as ˜10 ag of PrPᵣₑₛ in 0.5 mL of human plasma.

FIG. 16. Sensitivity of detection of sCJD brain homogenate spiked into cerebrospinal fluid (CSF) by e-QuIC. The assay detected sCJD brain tissue dilutions containing as little as ˜10 ag of PrPres in 0.5 ml of human cerebrospinal fluid.

FIG. 17. Sensitivity of detection of mouse-adapted RML scrapie brain homogenate spiked into plasma by e-QuIC. The assay detected down to 10⁻¹³ RML scrapie brain tissue dilutions (containing ˜100 ag of PrP-res) in 0.5 ml of plasma.

FIG. 18. eQuIC detection of endogenous PrPres in plasma of scrapie positive wild type (WT) & GPI— mice. The assay detected endogenous PrP-res in plasma from a wild-type mouse and a transgenic mouse expressing only PrP-sen that lacks the glycophosphatidylinositol anchor (GPI⁻). No prions were detected in a plasma sample from a non-infected wild type normal mouse.

### SEQUENCE LISTING

The nucleic and amino acid sequences listed are shown using standard letter abbreviations for nucleotide bases, and three letter code for amino acids, as defined in 37 C.F.R. 1.822. For nucleic acid sequences, only one strand of each nucleic acid sequence is shown, but the complementary strand is understood as included by any reference to the displayed strand.

SEQ ID NO: 1 is an amino acid sequence of a recombinant Syrian golden
hamster proteinase K-sensitive prion protein.
KKRPKPGGWNTGGSRYPGQGSPGGNRYPPQGGGTWGQPHGGGWGQPHGGGWGQPHGGGWGQ
PHGGGWGQGGGTHNQWNKPSKPKTNMKHMAGAAAAGAVVGGLGGYMLGSAMSRPMMHFGN
DWEDRYYRENMNRYPNQVYYRPVDQYNNQNNFVHDCVNITIKQHTVTTTTKGENFTETDIKIME
RVVEQMCTTQYQKESQAYYDGRRS
SEQ ID NO: 2 is an amino acid sequence of a recombinant mouse (Prnp-a)
proteinase K-sensitive prion protein.
KKRPKPGG WNTGGSRYPG QGSPGGNRYP PQGGTWGQPH GGGWGQPHGG SWGQPHGGSW
GQPHGGGWGQ GGGTHNQWNK PSKPKTNLKH VAGAAAAGAV VGGLGGYMLG
SAMSRPMIHF GNDWEDRYYR ENMYRYPNQV YYRPVDQYSN QNNFVHDCVN ITIKQHTVTT
TTKGENFTET DVKMMERVVE QMCVTQYQKE SQAYYDGRRS
SEQ ID NO: 3 is an amino acid sequence of a recombinant human (129M)
proteinase K-sensitive prion protein.
KKRPKPGG WNTGGSRYPG QGSPGGNRYP PQGGGGWGQP HGGGWGQPHG GGWGQPHGGG
WGQPHGGGWG QGGGTHSQWN KPSKPKTNMK HMAGAAAAGA VVGGLGGYML
GSAMSRPIIH FGSDYEDRYY RENMHRYPNQ VYYRPMDEYS NQNNFVHDCV NITIKQHTVT
TTTKGENFTE TDVKMMERVV EQMCITQYER ESQAYYQRGS S
SEQ ID NO: 4 is an amino acid sequence of a recombinant human (129V)
proteinase K-sensitive prion protein.
KKRPKPGG WNTGGSRYPG QGSPGGNRYP PQGGGGWGQP HGGGWGQPHG GGWGQPHGGG
WGQPHGGGWG QGGGTHSQWN KPSKPKTNMK HMAGAAAAGA VVGGLGGYVL
GSAMSRPIIH FGSDYEDRYY RENMHRYPNQ VYYRPMDEYS NQNNFVHDCV NITIKQHTVT
TTTKGENFTE TDVKMMERVV EQMCITQYER ESQAYYQRGS S
SEQ ID NO: 5 is an amino acid sequence of a recombinant bovine (6-
octarepeat) proteinase K-sensitive prion protein.
KKRPKP GGGWNTGGSR YPGQGSPGGN RYPPQGGGGW GQPHGGGWGQ PHGGGWGQPH
GGGWGQPHGG GWGQPHGGGG WGQGGTHGQW NKPSKPKTNM KHVAGAAAAG
AVVGGLGGYM LGSAMSRPLI HFGSDYEDRY YRENMHRYPN QVYYRPVDQY SNQNNFVHDC
VNITVKEHTV TTTTKGENFT ETDIKMMERV VEQMCITQYQ RESQAYYQRG AS
SEQ ID NO: 6 is an amino acid sequence of a recombinant ovine (136A
154R 171Q) proteinase K-sensitive prion protein.
154R 171Q) proteinase K-sensitive prion protein.
KKRPKP GGGWNTGGSR YPGQGSPGGN RYPPQGGGGW GQPHGGGWGQ PHGGGWGQPH
GGGWGQPHGG GGWGQGGSHS QWNKPSKPKT NMKHVAGAAA AGAVVGGLGG
YMLGSAMSRP LIHFGNDYED RYYRENMYRY PNQVYYRPVD QYSNQNNFVH DCVNITVKQH
TVTTTTKGEN FTETDIKIME RVVEQMCITQ YQRESQAYY RGAS
SEQ ID NO: 7 is an amino acid sequence of a recombinant Deer (96G
132M 138S) proteinase K-sensitive prion protein.
KKRPKP GGGWNTGGSR YPGQGSPGGN RYPPQGGGGW GQPHGGGWGQ PHGGGWGQPH
GGGWGQPHGG GGWGQGGTHS QWNKPSKPKT NMKHVAGAAA AGAVVGGLGG
YMLGSAMSRP LIHFGNDYED RYYRENMYRY PNQVYYRPVD QYNNQNTFVH DCVNITVKQH
TVTTTTKGEN FTETDIKMME RVVEQMCITQ YQRESQAYYQ RGAS
SEQ ID NO: 8 is an amino acid sequence of a full-length Syrian golden
hamster proteinase K-sensitive prion protein.
MANLSYWLLALFVAMWTDVGLCKK RPKPGGWNTG GSRYPGQGSP GGNRYPPQGG
GTWGQPHGGG WGQPHGGGWG QPHGGGWGQP HGGGWGQGGG THNQWNKPSK
PKTNMKHMAG AAAAGAVVGG LGGYMLGSAM SRPMMHFGND WEDRYYRENM
NRYPNQVYYR PVDQYNNQNN FVHDCVNITI KQHTVTTTTK GENFTETDIK IMERVVEQMC
TTQYQKESQA YYDGRRSSAV LFSSPPVILL ISFLIFLMVG
SEQ ID NO: 9 is an amino acid sequence of a full-length mouse (Prnp-a)
proteinase K-sensitive prion protein.
MANLGYWLLA LFVTMWTDVG LCKKRPKPGG WNTGGSRYPG QGSPGGNRYP PQGGTWGQPH
GGGWGQPHGG SWGQPHGGSW GQPHGGGWGQ GGGTHNQWNK PSKPKTNLKH
VAGAAAAGAV VGGLGGYMLG SAMSRPMIHF GNDWEDRYYR ENMYRYPNQV
YYRPVDQYSN QNNFVHDCVN ITIKQHTVTT TTKGENFTET DVKMMERVVE QMCVTQYQKE
SQAYYDGRRS SSTVLFSSPP VILLISFLIF LIVG
SEQ ID NO: 10 is an amino acid sequence of a full-length human (129M)
proteinase K-sensitive prion protein.
MANLGCWMLV LFVATWSDLG LCKKRPKPGG WNTGGSRYPG QGSPGGNRYP PQGGGGWGQP
HGGGWGQPHG GGWGQPHGGG WGQPHGGGWG QGGGTHSQWN KPSKPKTNMK
HMAGAAAAGA VVGGLGGYML GSAMSRPIIH FGSDYEDRYY RENMHRYPNQ VYYRPMDEYS
NQNNFVHDCV NITIKQHTVT TTTKGENFTE TDVKMMERVV EQMCITQYER ESQAYYQRGS
SMVLFSSPPV ILLISFLIFL IVG
SEQ ID NO: 11 is an amino acid sequence of a full-length human (129V)
proteinase K-sensitive prion protein.
MANLGCWMLV LFVATWSDLG LCKKRPKPGG WNTGGSRYPG QGSPGGNRYP PQGGGGWGQP
HGGGWGQPHG GGWGQPHGGG WGQPHGGGWG QGGGTHSQWN KPSKPKTNMK
HMAGAAAAGA VVGGLGGYVL GSAMSRPIIH FGSDYEDRYY RENMHRYPNQ VYYRPMDEYS
NQNNFVHDCV NITIKQHTVT TTTKGENFTE TDVKMMERVV EQMCITQYER ESQAYYQRGS
SMVLFSSPPV ILLISFLIFL IVG
SEQ ID NO: 12 is an amino acid sequence of a full-length chimeric
Hamster-Sheep (H-S) proteinase K-sensitive prion protein wherein residues
23-137 are of the Syrian hamster sequence and the remaining residues 138-231
were homologous to sheep residues 141-234 (R154, Q171 polymorph).
HMKKRPKPGGWNTGGSRYPGQGSPGGNRYPPQGGGTWGQPHGGGWGQPHGGGWGQPHG
GGWGQPHGGGWGQGGGTHNQWNKPSKPKTNMKHMAGAAAAGAVVGGLGGYMLGSAM
SRPLIHFGNDYEDRYYRENMYRYPNQVYYRPVDQYSNQNNFVHDCVNITVKQHTVTTTTK
GENFTETDIKIMERVVEQMCITQYQRESQAYYQRGAS.

### DETAILED DESCRIPTION OF SEVERAL EMBODIMENTS

The methods disclosed herein allow testing for prion contamination, diagnostics and/or surveillance in a number of biological samples, including blood, blood fractions, blood products, urine, nasal fluids, saliva, cerebral spinal fluid, feces, muscle biopsies, lymphoid tissues, skin samples, samples of tissues for transplantation, amongst others. These methods have medical and veterinary applications, and also can be used to test biotechnology products and environmental samples (such as water, soils, plants, landfills, sewage) and agriculture samples (such as animal-based foods, animal-based feeds & nutritional supplements, animal waste products, byproducts, carcasses, slaughterhouse wastes, specified risk materials) to ensure there is no contamination by prions. The presently disclosed methods also can be used for prion-free herd/flock certification, such as in cattle, sheep, and cervids. The methods disclosed herein can also be used to detect spontaneous Creutzfeldt-Jacob disease.

Currently, the most direct and reliable assay for the detection of TSE infectivity is animal bioassay. Quantification of infectivity can be achieved by end-point (Stamp et al., 1959) or limiting dilution bioassays (Gregori et al., 2004). For some combinations of prion agent and host species, strong correlations between infectivity titer and disease incubation period have been established in laboratory rodents, allowing the use of incubation period to measure infectivity levels (Hunter et al., 1963; Prusiner et al., 1982). The disadvantage of these bioassays is that they are animal-intensive, time-consuming and expensive. For certain murine-adapted scrapie strains, the cell culture based standard scrapie cell assay (SSCA) can also be used to measure infectivity levels by end-point and limiting dilution methods (Klohn et al., 2003). The SSCA offers several advantages over animal bioassays, but it still requires weeks to perform and has been limited to a few mouse-adapted scrapie strains. An analogous cell-based assay for cervid prions (designated CPCA) has also been reported (Bian et al., J Virol, 84, 8322-8326, 2010). The limitations of the animal bioassay, SSCA and CPCA, mean that more practical assays for prion quantitation are needed.

A number of highly sensitive in vitro methods for prion detection have been reported (Atarashi et al., Nat Methods, 4, 645-650, 2007; Atarashi et al., Nat Methods, 5, 211-212, 2008; Bieschke et al., Proc Natl Acad Sci USA, 97, 5468-5473, 2000; Chang et al., J Virol Methods, 159, 15-222009; Colby et al., Proc Natl Acad Sci USA, 104, 9741-9746, 2007; Fujihara et al., FEBS J, 276, 2841-2848, 2009; Orru et al., Protein Eng Des Sel, 22, 515-521, 2009; Rubenstein et al., J Gen Virol, 91, 1883-1892, 2010; Saa et al., Science, 313, 92-94, 2006a; Saa et al., J Biol Chem, 281, 35245-35252, 2006b; Terry et al., J Virol, 83, 12552-12558, 2009; Trieschmann et al., BMC Biotechnol, 5, 26, 2005; Wilham et al., PLoS Pathog, 6, e1001217, 2010). Fluorescence correlation spectroscopy can be used to detect femtomolar concentrations of PrP-res aggregates in cerebral spinal fluid (CSF) samples treated with fluorescently tagged antibodies (Bieschke et al., supra, 2000). The binding of fluorescently labeled recombinant PrP^{C} (rPrP^{c}) to synthetic prion protein aggregates allowed their ultra-sensitive detection by FACS analyses and a similar approach allowed the discrimination of sera of several BSE-infected and non-infected cattle (Trieschmann et al., 2005). Using the protein misfolding cyclic amplification (PMCA) reactions in multi-round sonicated reactions using brain-derived PrP^{C} as a substrate, as little as 1 ag of PrP-res can be detected (Saa et al., supra, 2006b). Coupling of limited serial PMCA with highly sensitive fluorescence detection technique called surround optical fiber immunoassay (SOPHIA) allows more rapid detection of as little as 10 ag PrP-res and discrimination of prion-infected versus uninfected blood samples (Chang et al., supra, 2009; Rubenstein et al., J Gen Virol, 91, 1883-1892, 2010).

The speed and practicality of PMCA assays has also been improved by the use of rPrP^{C} (Atarashi et al., supra, 2007) and by substituting shaking for the sonication step as described for the quaking-induced conversion (QuIC) reactions (Atarashi et al., supra, 2008; Orru et al., supra, 2009). The standard QuIC (also called “SQ”) assay can detect sub-femtogram amounts of PrP-res (less than one lethal intracerebral dose) in hamster brain homogenates (BH) within a single day. The effectiveness of the SQ for prion detection was demonstrated by its ability to discriminate normal from prion-infected hamsters using 2-μl samples of CSF (Atarashi et al., supra, 2008; Orru et al., supra, 2009) or nasal lavage (Bessen et al., PLoS Pathogens, 6, e1000837, 2010). Adaptations of SQ reactions have led to the sensitive detection of variant CJD (vCJD) in human tissue and scrapie in sheep tissue (Orru et al., supra, 2009).

The readout for SQ and PMCA assays is the detection of specific protease-resistant prion-seeded rPrP products by immunoblotting, which is difficult to adapt to automated high-throughput formats. An alternative, and potentially higher-throughput approach was used for the amyloid seeding assay (ASA) in which the fluorescent dye thioflavin T (ThT) was used to detect prion seeding of rPrP^{C} polymerization (Colby et al., Proc Natl Acad Sci USA, 104, 20914-20919, 2007, incorporated herein by reference). The ASA can also detect protease sensitive disease-causing prions and has a 98% correlation with neuropathological signs of prion disease (Colby et al., PLoS Pathog, 6, e1000736, 2010). However, a potentially confounding aspect of ASA is the frequent spontaneous formation of rPrP fibrils (without seeding by prions) within about twice the lag phase of prion-seeded reactions (Colby et al., Proc Natl Acad Sci USA, 104, 20914-20919, 2007). The problem of spontaneous fibril formation is greatly reduced in another prion-seeded rPrP^{c} polymerization assay, real-time (RT)-QuIC (also called RTQ, see, for example, Wilham et al., PLoS Pathog, 6, e1001217, 2010, which describes the assay and is incorporated herein by reference) which combines several aspects of the SQ assay (intermittent shaking, rPrP^{C} preparation, sample preparation, and a lack of chaotropic salts) with a fluorescent ThT readout like that of the ASA.

Until recently, a major limitation of the PMCA, SQ, RTQ and ASA methods was the lack of prion quantitation. Chen and colleagues reported a method called quantitative PMCA (qPMCA) in which PrP^{Sc} content is estimated by the number of PMCA rounds necessary for a positive response (Chen et al., Nat Methods, 7, 519-520, 2010). More recently, a different approach, using end-point dilution titration, was described in conjunction with the RTQ as a method for determining relative prion quantitation with in vitro prion seeding assays (Wilham et al., supra, 2010). Moreover, prion seeding activity was measured in the nasal fluids and CSF of prion-infected hamsters. Thus, in conjunction with the end-point dilution analysis, the RTQ can rapidly determine relative prion concentrations with a sensitivity that rivals that of animal bioassays, but with greatly reduced time and cost.

The presently described methods substantially improve the sensitivity and applicability of prion seeding/amplification assays such as the SQ and RTQ, in part by integrating them with novel prion/PrP-res/PrP^{Sc} immunoprecipitation and treatment protocols. The methods enable the capture and detection of extremely low levels of prions in various fluids or tissue extracts, including complex biological specimens such as blood plasma, which can contain strong inhibitors of prions.

### TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Aggregate: More than one molecule in association, such as dimers, multimers, and polymers of prion proteins, for instance aggregates, dimers, multimers, and polymers of PrP-res or rPrP-res^{(Sc)}.

Agitation: Introducing any type of turbulence or motion into a mixture or reaction mix, for examples by sonication, stirring, or shaking. In some embodiments, agitation includes the use of force sufficient to fragment rPrP-res^{(Sc)} aggregates, which disperses rPrP-res^{(Sc)} aggregates and/or polymers to facilitate further amplification. In some examples fragmentation includes complete fragmentation, whereas in other examples, fragmentation is only partial, for instance, a population of aggregates can be about 1%, 2%, 5%, 10%, 20%, 30%, 40%, 50%, 60%, 70%, 80%, 90%, or 100% fragmented by agitation. Exemplary agitation methods are described in the Examples section below.

Antibody: A polypeptide ligand comprising at least a light chain or heavy chain immunoglobulin variable region which specifically recognizes and binds an epitope of an antigen or a fragment thereof. An antibody can specifically bind PrP-res/PrP^{Sc}. Antibodies can be composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

The term antibody includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds an antigen of interest has a specific V_{H} region and the V_{L} region sequence, and thus specific CDR sequences. Antibodies with different specificities (due to different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected, or a progeny thereof. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Antibody binding affinity: Affinity of an antibody for an antigen, such as PrP-res. In one embodiment, affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity is at least about 1×10⁻⁸ M. In other embodiments, a high binding affinity is at least about 1.5×10⁻⁸M, at least about 2.0×10⁻⁸M, at least about 2.5×10⁻⁸M, at least about 3.0×10⁻⁸M, at least about 3.5×10⁻⁸M, at least about 4.0×10⁻⁸M, at least about 4.5×10⁻⁸M, or at least about 5.0×10⁻⁸ M.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T-cells respond. In one embodiment, T-cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and 2-dimensional nuclear magnetic resonance. An antigen can be a tissue-specific antigen, or a disease-specific antigen, such as PrP-res. These terms are not exclusive, as a tissue-specific antigen can also be a disease specific antigen.

Conservative variant: In the context of a prion protein, refers to a peptide or amino acid sequence that deviates from another amino acid sequence only in the substitution of one or several amino acids for amino acids having similar biochemical properties (so-called conservative substitutions). Conservative amino acid substitutions are likely to have minimal impact on the activity of the resultant protein. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. In some examples, prion protein variants can have no more than 1, 2, 3, 4, 5, 10, 15, 30, 45, or more conservative amino acid changes.

In one example, a conservative variant prion protein is one that functionally performs substantially like a similar base component, for instance, a prion protein having variations in the sequence as compared to a reference prion protein. For example, a prion protein or a conservative variant of that prion protein, will aggregate with PrP-res (or PrP^{Sc}), for instance, and will convert rPrP^{C} to rPrP-res^{(Sc)} (or will be converted to rPrP-res^{(Sc)}). In this example, the prion protein and the conservative variant prion protein do not have the same amino acid sequences. The conservative variant can have, for instance, one variation, two variations, three variations, four variations, or five or more variations in sequence, as long as the conservative variant is still complementary to the corresponding prion protein.

In some embodiments, a conservative variant prion protein includes one or more conservative amino acid substitutions compared to the prion protein from which it was derived, and yet retains prion protein biological activity. For example, a conservative variant prion protein can retain at least 10% of the biological activity of the parent prion protein molecule from which it was derived, or alternatively, at least 20%, at least 30%, or at least 40%. In some preferred embodiments, a conservative variant prion protein retains at least 50% of the biological activity of the parent prion protein molecule from which it was derived. The conservative amino acid substitutions of a conservative variant prion protein can occur in any domain of the prion protein.

Contacting: “Contacting” includes in solution and solid phase, for example contacting a sample with a specific binding agent, such as an antibody that specifically binds PrP-res.

Conditions sufficient to detect: Any environment that permits the desired activity, for example, that permits an antibody to bind an antigen, such as PrP-res, and the interaction to be detected. For example, such conditions include appropriate temperatures, buffer solutions, and detection means such as and digital imaging equipment.

Detect: To determine if an agent (such as a signal or protein, for example PrP-res) is present or absent. In some examples, this can further include quantification, for example the quantification of the amount of PrP-res in a sample, such as a serum sample, or a fraction of a sample.

Diagnostic: Identifying the presence or nature of a pathologic condition, such as, but not limited to, identifying the presence of PrP-res, such as in Creutzfeldt-Jacob disease. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (for example severity) of a pathologic condition.

Disaggregate: To partially or complete disrupt an aggregate, such as an aggregate of PrP-res or rPrP-res^{(Sc)}.

Encode: Any process whereby the information in a polymeric macromolecule or sequence is used to direct the production of a second molecule or sequence that is different from the first molecule or sequence. As used herein, the term is construed broadly, and can have a variety of applications. In some aspects, the term “encode” describes the process of semi-conservative DNA replication, wherein one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (for instance, by the process of transcription incorporating a DNA-dependent RNA polymerase enzyme). Also, an RNA molecule can encode a peptide, as in the process of translation. When used to describe the process of translation, the term “encode” also extends to the triplet codon that encodes an amino acid. In some aspects, an RNA molecule can encode a DNA molecule, for instance, by the process of reverse transcription incorporating an RNA-dependent DNA polymerase. In another aspect, a DNA molecule can encode a peptide, where it is understood that “encode” as used in that case incorporates both the processes of transcription and translation.

Fluorophore: A chemical compound, which when excited by exposure to a particular stimulus, such as a defined wavelength of light, emits light (fluoresces), for example at a different wavelength (such as a longer wavelength of light). Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) can eliminate the need for an external source of electromagnetic radiation, such as a laser.

Examples of particular fluorophores that can attached to antibodies that specifically binds PrP^{Sc} are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcoumarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives; LightCycler Red 640; Cy5.5; and Cy56-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC® (Applied Biosystems), LC Red 640, LC Red 705, Yakima yellow amongst others.

Other suitable fluorophores include those known to those skilled in the art, for example those available from Molecular Probes (Eugene, Oreg.). In particular examples, a fluorophore is used as a donor fluorophore or as an acceptor fluorophore. In some examples, a fluorophore is detectable label, such as a detectable label attached to an antibody.

Immunoassay: A biochemical test that measures the presence or concentration of a substance in a sample, such as a biological sample, for example a serum sample obtained from a subject, using the reaction of an antibody to its cognate antigen, for example the specific binding of an antibody to a protein, such PrP-res. Both the presence of antigen or the amount of antigen present can be measured. In some examples, the amount of PrP-res is measured.

Immunoprecipitation (IP): The technique of precipitating a protein antigen out of solution using an antibody or peptides that specifically binds to that particular protein. These solutions will often be in the form of a crude lysate of an animal tissue. Other sample types could be body fluids or other samples of biological origin. Generally, in IP the antibody or peptides are coupled to a solid substrate at some point in the procedure.

Isolated: An “isolated” biological component, such as a peptide or assembly of polypeptides (for example PrP^{Sc}), cell, nucleic acid, or serum samples has been substantially separated, produced apart from, or purified away from other biological components in the cell of the organism in which the component naturally occurs, for instance, other chromosomal and extrachromosomal DNA and RNA, and proteins. Nucleic acids, peptides and proteins that have been “isolated” thus include nucleic acids and proteins purified by standard purification methods. The term also embraces nucleic acids, peptides and proteins prepared by recombinant expression in a cell as well as chemically synthesized peptide and nucleic acids. The term “isolated” or “purified” does not require absolute purity; rather, it is intended as a relative term. Thus, for example, an isolated peptide preparation is one in which the peptide or protein is more enriched than the peptide or protein is in its natural environment within a cell. Preferably, a preparation is purified such that the protein or peptide represents at least 50% of the total peptide or protein content of the preparation, such as at least 50%, at least 60%, at least 70%, at least 80%, at least 90%, at least 95%, or even at least 99% of the peptide or protein concentration.

Nucleic acid molecule: A polymeric form of nucleotides, which can include both sense and anti sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single and double stranded forms of DNA. A nucleic acid molecule can include either or both naturally occurring and modified nucleotides linked together by naturally occurring and/or non naturally occurring nucleotide linkages.

Prion: A type of infectious agent composed mainly of protein. Prions cause a number of diseases in a variety of animals, including bovine spongiform encephalopathy (BSE, also known as mad cow disease) in cattle and Creutzfeldt-Jakob disease in humans. All known prion diseases affect the structure of the brain or other neural tissue, and all are untreatable and fatal. The “transmissible spongiform encephalopathies (TSEs)” or prion diseases are fatal neurodegenerative disorders that include human Creutzfeldt-Jakob disease (CJD), bovine spongiform encephalopathy (BSE), sheep scrapie, cervid chronic wasting disease (CWD), and transmissible mink encephalopathy (TME).

Prions are believed to infect and propagate by refolding abnormally into a structure that is able to convert normal molecules of the protein into the abnormally structured forms (for instance, PrP^{Sc} in scrapie or PrP^{vCJD} in variant CJD), which are usually partially resistant to proteinase K digestion, and hence will be designated generically herein as PrP-res for PrP-resistant. Most, if not all, known prions can polymerize into amyloid fibrils rich in tightly packed beta sheets. This altered structure renders them unusually resistant to denaturation by chemical and physical agents, making disposal and containment of these particles difficult.

In prion diseases, the pathological, typically protease-resistant form of prion protein, PrP-res, appears to propagate itself in infected hosts by inducing the conversion of its normal host-encoded protease-sensitive precursor, PrP-sen or PrP^{C}, which is sensitive to proteinase K digestion, into PrP-res. PrP-sen (PrP^{C}) is a monomeric glycophosphatidylinositol-linked glycoprotein that is low in β-sheet content, and highly protease-sensitive. Conversely, PrP-res (e.g. PrP^{Sc}) aggregates are high in β-sheet content and partially protease-resistant. Mechanistic details of the conversion are not well understood, but involve direct interaction between PrP-res and PrP^{C}, resulting in conformational changes in PrP^{C} as the latter is recruited into the growing PrP-res multimer (reviewed in Caughey & Baron, Nature 443, 803-810, 2006). Accordingly, the conversion mechanism has been tentatively described as autocatalytic seeded (or nucleated) polymerization. In the assays disclosed herein, addition of a biological sample comprising PrP-res or prions results in the conversion of recombinant PrP^{C} (rPrP^{C}) into rPrP-res^{(Sc)} in a reaction mixture which can then be detected. The recombination protein, rPrP-res^{(Sc)} is a generic term for the prion-induced rPrP conversion product, regardless of the species and strain of origin of the prions. The recombinant protein, rPrP-res^{(Sc)}, is not infectious.

PMCA or Protein Misfolding Cyclic Amplification: A method for amplifying PrP-res in a sample by mixing PrP^{C} with the sample, incubating the reaction mix to permit PrP-res to initiate the conversion of PrP^{C} to aggregates of PrP-res, fragmenting any aggregates formed during the incubation step (typically by sonication), and repeating one or more cycles of the incubation and fragmentation steps.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell.

QuIC or Quaking Induced Conversion: A particular type of PrP amplification assay, in which shaking of the reaction vessels is performed instead of sonication to disrupt aggregated rPrP^{C} and rPrP-res^{(Sc)}.

Real Time (RT)-QuIC: An assay that includes intermittent shaking to disrupt aggregated PrP^{C} and PrP-res and includes the use of a fluorescent readout, such as the fluorescent dye thioflavin T (ThT). Exemplary protocols are disclosed, for example, in Wilham et al., PLOS Pathog. 6(12): e1001217, pages 1-15. Generally, this assay uses PrP^{C} as a substrate, intermittently shaken reactions, predominantly detergent-free (such as ≦0.002% of SDS) or detergent-free, and chaotrope-free reactions conditions, and ThT-based fluorescent detections of prion seeded rPrP^{C} amyloid fibrils.

Sample: A biological sample obtained from a subject, such as a human or veterinary subject, which contains for example nucleic acids and/or proteins. As used herein, biological samples include all clinical samples useful for detection of PrP-res/prions in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In particular embodiments, the biological sample is obtained from a subject, such as in the form of a blood sample, such as serum sample. Samples also include environmental samples, such as soil or water samples.

Sequence identity: The similarity between two nucleic acid sequences or between two amino acid sequences is expressed in terms of the level of sequence identity shared between the sequences. Sequence identity is typically expressed in terms of percentage identity; the higher the percentage, the more similar the two sequences. Methods for aligning sequences for comparison are described in detail below, in section IV E of the Detailed Description.

Single Round: Performing a method wherein serial amplification is not performed. For example, rPrP-res^{(Sc)} can be amplified in a sample, by mixing the sample with purified rPrP^{C} to make a reaction mix; performing an amplification reaction that includes (i) incubating the reaction mix to permit coaggregation of the rPrP^{C} with the PrP-res that may be present in the reaction mix, and maintaining incubation conditions that promote coaggregation of the rPrP^{C} with the PrP-res and results in a conversion of the rPrP^{C} to rPrP-res^{(Sc)} while inhibiting development of rPrP-res^{(spon)} (protease-resistant rPrP products that are generated spontaneously in the absence of prions or PrP-res) (ii) agitating aggregates formed during step (i); (iii) optionally repeating steps (i) and (ii) one or more times. rPrP-res^{(Sc)} is detected in the reaction mix, wherein detection of rPrP-res^{(Sc)} in the reaction mix indicates that PrP-res was present in the sample. Additional substrate (rPrP^{C}) can be added during the reaction, such as during the lag phase (between the addition of the sample and the formation of detectable of rPrP-res^{(Sc)}). However, a portion of the reaction mix is not removed and incubated with additional rPrP^{C} in a separate reaction mixture.

Sonication: The process of disrupting or dispersing biological materials using sound wave energy.

Specific binding agent: An agent that binds substantially only to a defined target. In some embodiments, a specific binding agent is an antibody that specifically binds PrP-res but not PrP^{C}.

The term “specifically binds” refers to the preferential association of an antibody or other ligand, in whole or part, with an antigen. Specific binding may be distinguished as mediated through specific recognition of the antigen. Although selectively reactive antibodies bind antigen, they may do so with low affinity. On the other hand, specific binding results in a much stronger association between the antibody (or other ligand) and antigen (or cells bearing the antigen) than between the bound antibody (or other ligand) and another protein (or cells lacking the antigen). Specific binding typically results in greater than 2-fold, such as greater than 5-fold, greater than 10-fold, or greater than 100-fold increase in amount of bound antibody or other ligand (per unit time) to a cell or tissue expressing the target epitope as compared to a cell or tissue lacking this epitope. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Suitable methods and materials for the practice or testing of the disclosure are described below. However, the provided materials, methods, and examples are illustrative only and are not intended to be limiting. Accordingly, except as otherwise noted, the methods and techniques of the present disclosure can be performed according to methods and materials similar or equivalent to those described and/or according to conventional methods well known in the art and as described in various general and more specific references that are cited and discussed throughout the present specification (see, for instance, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999).

The methods disclosed herein utilize affinity purification, such as immunoprecitiation of prion proteins, followed by another detection method, such as, but not limited to, a quaking induced conversion assay (QuIC) or a Real-Time quaking induced conversion assay (RT-QuIC) to detect prions in a sample, such as a biological sample. The methods disclosed herein allow the testing for prion contamination, diagnostics and/or surveillance in a number of biological samples, including blood, blood fractions, and blood products, urine, nasal fluids, saliva, cerebral spinal fluid, feces, muscle biopsies, lymphoid tissues, skin samples, samples of tissues for transplantation, amongst others. These methods have both medical/veterinary applications, and also can be used to test biotechnology products and environmental samples (such as water, soils, plants, landfills, sewage) and agriculture samples (such as animal-based foods, animal-based feeds & nutritional supplements, animal waste products, byproducts, carcasses, slaughterhouse wastes, specified risk materials) to ensure there is no contamination by prions. The presently disclosed methods also can be used for prion-free herd/flock certification, such as in cattle, sheep, and cervids. The combination of affinity purification (such as immunoprecipitation) and QuIC or RT-QuIC provide an unexpectedly superior sensitivity and specificity for the detection of PrP-res.

I. Overview of Prions and Prion Disease

The transmissible spongiform encephalopathies (TSEs, or prion diseases) are infectious neurodegenerative diseases of mammals that include (but are not limited to) scrapie in sheep, bovine spongiform encephalopathy (BSE; also known as mad cow disease) in cattle, transmissible mink encephalopathy (TME) in mink, chronic wasting disease (CWD) in elk, moose, and deer, feline spongiform encephalopathy in cats, exotic ungulate encephalopathy (EUE) in nyala, oryx and greater kudu, and Creutzfeldt-Jakob disease (CJD) and its varieties (iatrogenic Creutzfeldt-Jakob disease (iCJD), variant Creutzfeldt-Jakob disease (vCJD), familial Creutzfeldt-Jakob disease (fCJD), and sporadic Creutzfeldt-Jakob disease (sCJD)), Gerstmann-Sträussler-Scheinker syndrome (GSS), fatal familial insomnia (WI), sporadic fatal insomnia (sFI), and kuru in humans. TSEs have incubation periods of months to years, but after the appearance of clinical signs often are rapidly progressive, untreatable, and invariably fatal. Attempts at TSE risk reduction have led to profound changes in the production and trade of agricultural goods, medicines, cosmetics, and biotechnology products.

In TSEs the pathological, protease-resistant form of prion protein, termed PrP^{Sc} or PrP-res, appears to propagate itself in infected hosts by inducing the conversion of its normal host-encoded precursor, PrP-sen, also known as PrP, into PrP-res. PrP^{C} is a monomeric glycophosphatidylinositol-linked glycoprotein that is low in β-sheet content, and highly protease-sensitive. Conversely, PrP-res aggregates are high in β-sheet content and partially protease-resistant. Mechanistic details of the conversion are not well understood, but involve direct interaction between PrP-res and PrP, resulting in conformational changes in PrP^{C} as the latter is recruited into the growing PrP-res multimer (reviewed in Caughey & Baron (2006) Nature 443, 803-810). Accordingly, the conversion mechanism has been tentatively described as autocatalytic seeded (or nucleated) polymerization.

To better understand the mechanism of prion propagation, many attempts to recapitulate PrP-res formation in cell-free systems have been made. Initial experiments showed that PrP-res can induce the conversion of PrP^{C} to PrP-res-like products with strain- and species-specificities, albeit with substoichiometric yields. More recently, it was shown that PrP-res formation and TSE infectivity can be amplified indefinitely in crude brain homogenates, a medium containing numerous potential cofactors for conversion (Castilla et al., (2005) Cell 121, 195-206). Dissection of this “protein misfolding cyclic amplification” (PMCA) reaction showed that PrP-res and prion infectivity also could be amplified using PrP^{C} purified from brain tissue as long as polyanions such as RNA were added (Deleault et al., (2007) Proc Natl Acad Sci USA. 104(23):9741-6). Recombinant PrP^{C} (rPRP^{C}, also called rPrP-sen) from E. coli lacks glycosylation and the GPI anchor can be induced to polymerize into amyloid fibrils spontaneously or when seeded by preformed rPrP fibrils. Although most rPrP amyloid preparations are not infectious, some preparations composed of rPrP^{C} alone, or in combination with lipids and nucleic acids have at least modest amounts of infectivity [Legname et al. (2004) Science 305, 673-676; Kim et al., (2010) J Biol Chem 285(19):14083-7; Wang et al., (2010) Science 327(5969):1132-5; Makarava et al., (2010) Acta Neuropath 119(2):177-87; Colby et al., (2010) PLoS Path 6(1):e1000736].

A key challenge in coping with TSEs is the rapid detection of low levels of TSE infectivity (prions) by rapid methods. The most commonly used marker for TSE infections is PrP-res, and the PMCA reaction allows extremely sensitive detection of PrP-res at levels below single infectious units in infected tissue. However, as previously noted, current limitations of PMCA include the time required to achieve optimal sensitivity (−3 weeks) and the use of brain PrP^{C} as the amplification substrate.

The most common TSE in animals is scrapie, but the most famous and dangerous TSE is BSE, which affects cattle and is known by its lay term “mad cow disease.” In humans, the most common TSE is CJD, which occurs worldwide with an incidence of 0.5 to 1.5 new cases per one million people each year. Three different forms of CJD have been traditionally recognized: sporadic (sCJD; 85% of cases), familial (fCJD; 10%), and iatrogenic (iCJD; 5%). However, in 1996, a new variant form of CJD (vCJD) emerged in the UK that was associated with consumption of meat infected with BSE. In contrast with typical sCJD, vCJD affects young patients with an average age of 27 years, and causes a relatively long illness (14 months compared with 4.5 months for sCJD). Because of insufficient information available about the incubation time and the levels of exposure to contaminated cattle food products, it is difficult to predict the future incidence of vCJD. In animals, there is little evidence for inherited forms of the disease, and most cases appear to be acquired by horizontal or vertical transmission.

The clinical diagnosis of sCJD is based on a combination of rapidly progressive multifocal dementia with pyramidal and extrapyramidal signs, myoclonus, and visual or cerebellar signs, associated with a characteristic periodic electroencephalogram (EEG). A key diagnostic feature of sCJD that distinguishes it from Alzheimer's disease and other dementias is the rapid progression of clinical symptoms and the short duration of the disease, which is often less than 2 years. The clinical manifestation of fCJD is very similar, except that the disease onset is slightly earlier than in sCJD. Family history of inherited CJD or genetic screening for mutations in the prion protein gene are used to establish fCJD diagnosis, although lack of family history does not exclude an inherited origin.

Variant CJD appears initially as a progressive neuropsychiatric disorder characterized by symptoms of anxiety, depression, apathy, withdrawal and delusions, combined with persistent painful sensory symptoms and followed by ataxia, myoclonus, and dementia. Variant CJD is differentiated from sCJD by the duration of illness (usually longer than 6 months) and EEG analysis (vCJD does not show the atypical pattern observed in sCJD). A high bilateral pulvinar signal noted during MRI is often used to help diagnose vCJD. In addition, a tonsil biopsy can be used to help diagnose vCJD, based on a number of cases of vCJD have been shown to test positive for PrP^{vCJD} staining in lymphoid tissue (such as tonsil and appendix). However, because of the invasive nature of this test, it is performed only in patients who fulfill the clinical criteria of vCJD where the MRI of the brain does not show the characteristic pulvinar sign.

GSS is a dominantly inherited illness that is characterized by dementia, Parkinsonian symptoms, and a relatively long duration (typically, 5-8 years). Clinically, GSS is similar to Alzheimer's disease, except that is often accompanied by ataxia and seizures. Diagnosis is established by clinical examination and genetic screening for prion protein mutations. FFI is also dominantly inherited and associated with prion protein mutations. However, the major clinical finding associated with FFI is insomnia, followed at late stages by myoclonus, hallucinations, ataxia, and dementia.

A need remains to be able to detect the TSEs, including CJD, as well as to be able to detect prions in biological and environmental samples, including but not limited to detecting prion contamination in the blood products. Thus, there is a need for a rapid and sensitive assay for the detection of prions. The present disclosure provides an assay system that uses immunoprecipitation, followed by a second detection method, such as QuIC and RT-QuIC, to provide a sensitive and specific method for detecting prions. In some embodiments, pre-emptive replenishment of rPrPC substrate in the QuIC or RT-QuIC assay is utilized. Without being bound by theory, this combined assay provides unanticipated increases in sensitivity, and surprising reductions in assay time. These assays allow the capture and detection of prions from extremely low-titered (for example, 0.001 infectious unit per ml) and/or inhibitor-laden fluids such as blood plasma in short periods, such as within two days.

II. Immunoprecipitation

The methods disclosed herein include contacting a sample, such as a biological sample, with an antibody that specifically binds only the disease related conformation of a prion protein (e.g. PrP^{Sc}, PrP^{vCJD} or PrP-res). In the methods disclosed herein, the sample, such as a biological sample, is contacted with a capture-monoclonal antibody (or epitope-binding fragment thereof), which can be immobilized on a solid substrate. Monoclonal antibodies can be selected that specifically bind an epitope that is expressed on PrP-res, but not on PrP.

The monoclonal antibodies that specifically bind PrP-res or PrP^{Sc} can be from any species, such as murine antibodies. The monoclonal antibodies can be produced by known monoclonal antibody production techniques. Typically, monoclonal antibodies are prepared by recovering spleen cells from immunized animals with the protein of interest and immortalizing the cells in conventional fashion, for example, by fusion with myeloma cells or by Epstein-Barr virus transformation, and screening for clones expressing the desired antibody. See, for example, Kohler and Milstein Eur. J. Immunol. 6:511 (1976). Monoclonal antibodies, or the epitope-binding region of a monoclonal antibody, may alternatively be produced by recombinant methods. Thus, in some embodiments, chimeric or humanized forms of a monoclonal antibody are utilized, wherein the antibody of use includes the complementarity determining regions (CDRs) of an antibody that specifically binds PrP-res or PrP^{Sc}.

By way of example, where the protein of interest is a prion protein that is capable of changing conformation to form PrP-res aggregates, the monoclonal antibody can be a murine monoclonal antibody that is generated by immunizing “knock out” mice with recombinant normal mouse cellular protein (PrP^{C}). Spleen cells (antibody producing lymphocytes of limited life span) from the immunized mice can then be fused with non-producing myeloma cells (tumor lymphocytes that are “immortal”) to create hybridomas. The hybridomas can then be screened for the production of antibody specific to PrP-res or PrP^{Sc} and the ability to be propagated in tissue culture. These hybridomas can then be cultured to provide a permanent and stable source for the specific monoclonal antibodies. Particular monoclonal antibodies produced by this method are disclosed in U.S. Pat. No. 6,528,269. These monoclonal antibodies include 2F8, 5B2, 6H3, 8C6, 8H4 and 9H7 produced by cell lines PrP2F8, PrP5B2, PrP6H3, PrP8C6, PrP8H4 and PrP9H7, that can specifically bind human PrP-res, and also bind PrP-res from mouse, cow, sheep and other species, see also U.S. Published Patent Application No. 2005/0118720, which is incorporated herein by reference.

The methods disclosed herein can also utilize monoclonal antibody 15B3, which is described in U.S. Published Patent Application No. 2008/0220447, published Sep. 1, 2008, which is incorporated herein by reference. The antibody 15B3 is available from Prionics AG, Zurich, Switzerland and methods to generate this antibody are disclosed in PCT Publication No. WO 98/37210, which is incorporated herein by reference. This PCT Publication also describes antibodies that bind PrP-res but not PrP. PCT Publication No. WO 98/37210 discloses that hybridomas that produce antibody 15B3 were deposited in accordance with the Budapest treaty at DSMZ—Deutsche Sammlung von Mikroorganismen and Zellkulturen GmbH (Germany) (Zellkulturen GmbH, Inhoffenstraβe 7 B 38124 Braunschweig, Germany) under Accession Number: DSM ACC2298.

The IgM monoclonal antibody 15B3 specifically recognizes the disease-associated form of the prion protein (i.e., PrP-res or PrP^{Sc}) and is capable of detecting abnormal PrP in brain homogenates without the need of PK digestion (Korth et al., Nature 1997; 390:74-77, 1997, herein incorporated by reference). In addition, the antibody 15B3 was shown to bind to protease-sensitive forms of PrP^{Sc} in a transgenic mouse model of Gerstmann Sträussler Scheinker syndrome which is of considerable importance as it was shown that infectivity in blood is sensitive to protease digestion (Nazor et al., EMBO J. 24(13):2472-80, 2005; Yakovleva et al., Transfusion 44:1700-5, 2004).

The capture-monoclonal antibody (such as 15B3, Ig 261, Ig W226 or 262) can be immobilized on a solid phase by insolubilizing the capture-monoclonal antibody before the assay procedure, as by adsorption to a water-insoluble matrix or surface (U.S. Pat. No. 3,720,760, herein incorporated by reference in its entirety) or non-covalent or covalent coupling, for example, using glutaraldehyde or carbodiimide cross-linking, with or without prior activation of the support with, e.g., nitric acid and a reducing agent (as described in U.S. Pat. No. 3,645,852 or in Rotmans et al., J. Immunol. Methods 57:87-98, 1983), or afterward, such as by immunoprecipitation.

The solid phase used for immobilization may be any inert support or carrier that is essentially water insoluble and useful in immunometric assays, including supports in the form of, for example, surfaces, particles, porous matrices, sepharose, etc. Examples of commonly used supports include small sheets, Sephadex, polyvinyl chloride, plastic beads, magnetic beads, and assay plates or test tubes manufactured from polyethylene, polypropylene, polystyrene, and the like including 96-well microtiter plates and 384-well microtiter well pates, as well as particulate materials, such as filter paper, agarose, cross-linked dextran, and other polysaccharides. Alternatively, reactive water-insoluble matrices, such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture-monoclonal antibody immobilization. In one example, the immobilized capture-monoclonal antibodies are coated on a microtiter plate, and in particular the solid phase can be a multi-well microtiter plate. For example, the multi-well microtiter plate can be a microtest 96-well ELISA plate. The solid phase can be a magnetic bead, such as DYNABEADS® (Invitrogen) or other magnetic beads, such as those available from NEW ENGLAND BIOLABS® or DYNAL® magnetic beads.

Generally, the capture-monoclonal antibody (such as, but not limited to, 15B3) is attached to the solid substrate. This attachment can be through a non-covalent or covalent interaction or physical linkage as desired. Techniques for attachment include those described in U.S. Pat. No. 4,376,110 and the references cited therein. If covalent binding is used, the plate, bead or other solid phase can be incubated with a cross-linking agent together with the capture reagent under conditions well known in the art.

The solid substrate can also have an antibody, such as a rabbit anti-mouse antibody or a rabbit anti-human antibody covalently linked to the solid substrate. The antibody attached to the solid substrate can then be incubated with a second antibody of interest (such as a mouse or human antibody) to achieve attachment of the second antibody to the solid substrate. In one specific non-limiting example, a rabbit anti-mouse antibody is coupled to the solid substrate, which is then incubated with a second antibody that specifically binds a prion protein, such as, but not limited to, 15B3, IgG W226 or IgG 261.

Commonly used cross-linking agents for attaching a capture-monoclonal antibody to the solid phase substrate include, for example 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents, such as methyl-3-[(p-azidophenyl)dithio]propioimidate yield photoactivatable intermediates capable of forming cross-links in the presence of light.

If micro-titer well plates (e.g., 96-well plates or 384-well plates) are utilized, they can be coated with affinity purified capture monoclonal antibodies (typically diluted in a buffer) at, for example, room temperature and for about 2 to about 3 hours. The plates can also be coated with the antibody that specifically binds PrP-res or PrP^{Sc} directly. The plates may be stacked and coated long in advance of the assay itself, and then the assay can be carried out simultaneously on several samples in a manual, semi-automatic, or automatic fashion, such as by using robotics.

Similarly, if DYNABEADS®, such as DYNABEADS® M-450 (rat anti-mouse IgM) are utilized, the beads can be coated with the antibody using any procedures known in the art. In one non-limiting examples, the DYNABEADS® are suspended in a vial using vortexing, and then an appropriate amount of the DYNABEADS®, is moved to a polypropylene or polystyrene tube. The tube is placed on a magnet for a short period of time, and then removed from the magnet. A coating buffer is added, and the beads are mixed, such as by using a vortex. In one non-limiting example, a coating buffer comprising about 0.01% to 1%, such as about 0.1% bovine serum albumin in phosphate buffered saline is utilized. Examples of additional blocking agents for the coating buffer might include, but are not limited to egg albumin, casein, and non-fat milk. The antibody of interest is added (such as, but not limited to, 15B3, IgG W226 or IgG 261), and the DYNABEADS® are incubated with the antibody of interest with gentle mixing for a sufficient time for the antibody to adhere to the beads. A magnet can then be used to separate the coated beads from the supernatant, and a coating buffer can be added. The DYNABEADS® coupled to the antibody can be washed repeatedly, and stored for future use.

In one example, the antibody (such as, but not limited to, 15B3) can be coupled to the substrate as about 5 μg antibody per 100 μl DYNABEADS®. In another example, the antibody (such as, but not limited to, 15B3) can be coupled to the substrate as per 1×10⁻⁶ DYNABEADS® per μg of 15B3 antibody. In another example, 2, 3, 4, 5, 6, 7, 8, 9, 10, 11, 12, 15, 20 or 30-fold more antibody can be utilized, such as 30-50 μg, such as 36 μg of antibody, for example 15B3 per 100 μl DYNABEADS® (for example, 4×10⁸ beads/ml). In other embodiments, ing to 10 μg of antibody can be used for 1×10⁸ beads. In yet another example, 100-300 μg of antibody, for example 15B3 per 1×10⁻⁸ DYNABEADS® (for example, 4×10⁸ beads/ml) can be utilized. In some non-limiting examples, the concentration of the antibody on the magnetic beads is about 10-500 μg of 15B3 per 1×10⁸ beads.

Coated plates or beads optionally can be treated with a blocking agent that binds non-specifically to and saturates the binding sites to prevent unwanted binding of the free ligand to the excess sites on the wells of the plate. Examples of appropriate blocking agents for this purpose include gelatin, bovine serum albumin, egg albumin, casein, and non-fat milk.

After coating and blocking, a sample to be analyzed is added to the immobilized antibody. The sample can be a biological sample or an environmental sample. The sample can be homogenized (such as for a tissue sample, such as a brain sample), and appropriately diluted with, for example, a lysis buffer (e.g., phosphate-buffered saline (PBS) with 1% Nonidet P-40, 0.5% sodium deoxycholate, 5 mM EDTA, and pH 8.0). Other detergents can be used, such as anionic, cationic or non-ionic detergents, including but not limited to sodium dodecyl sulfated (SDS) to homogenize a sample. Alternatively, mechanical means can be utilized, such as using pipetting or devices such as blenders and homogenizers. The biological sample can be a blood, serum, plasma, or a sample of another biological fluid, such as, but not limited to cerebral spinal fluid or nasal fluid. The sample can be a tissue sample, such as a brain sample or a lymphoid tissue sample (such as tonsils). The sample can be diluted, such as in buffer, for example a buffer including bovine serum albumin. In one embodiment, the sample is diluted in a buffer, such as tris buffered saline (TBS) or phosphate buffered saline (PBS), optionally including a detergent. The detergent can be a cationic, anionic or non-ionic detergent. In one embodiment the detergent is Sarkosyl. For example, the beads can be contacted with the sample in the presence of about 0.1% to about 1%, such as about 0.4% Sarkosyl in TBS. In another embodiment, the beads can be contacted with the sample in the presence of about 0.1% to about 1% Sarkosyl in TBS, such as 0.4% to about 1% Sarkosyl in a buffer, such as TBS or PBS. In some examples, about 0.1%, about 0.4%, about 1%, about 2%, about 3% or about 4% Sarkosyl in a buffer, such as TBS or PBS, is utilized.

For sufficient sensitivity, the amount of sample added to the immobilized capture monoclonal antibody can be such that the immobilized capture monoclonal antibodies are in molar excess of the maximum molar concentration of the conformational altered protein anticipated in the biological sample after appropriate dilution of the sample.

The conditions for incubation of the biological sample and immobilized monoclonal antibody are selected to maximize sensitivity of the assay and to minimize dissociation. Preferably, the incubation is accomplished at fairly constant temperatures, ranging from about 0° C. to about 40° C., such as at about 4° C., room temperature (e.g., about 25° C.), about 35° C. to about 39° C., or at about 37° C., or about 35° C. to 40° C. In some embodiments the temperature is about 19 to about 40° C., such as at room temperature. The time for incubation can be for example, 2 hours to 12 hours, such as overnight. In some examples, the incubation period is 2, 4, 6, 8, 10, 12, 20 or 24 hours, for example overnight at about 0° C. to about 40° C., such as at about 4° C., room temperature (e.g., about 25° C.), or 37° C. In specific non-limiting examples, the incubation is about 2 hours at room temperature or overnight at 4° C., such as about 12 hours at 4° C. or for about 10 to 20 hours at room temperature, such as 20 hours at room temperature or 37° C.

Following contact of the biological sample with the immobilized capture-monoclonal antibody (such as 15B3), the biological sample is washed. The washing medium is generally a buffer (“washing buffer”) with a pH determined using the considerations and buffers typically used for the incubation step. The washing may be done, for example, one, two, three or more times. The washing can be performed at any temperature, such as from about 0° C. to about 40° C., such as at room temperature (e.g., 25° C.) or at 37° C. In additional embodiments, the method comprises using SDS in a buffer, such as 0.01% to 0.1% SDS, such as about 0.01%, 0.02%, 0.03%, 0.04%, 0.05%, 0.06% or 0.07% SDS, for example 0.04% to 0.06% SDS, such as about 0.05% SDS. Examples of washing buffers include, but are not limited to, phosphate buffered saline (PBS) and Tris buffered saline (TBS), optionally including Sarkosyl, such as about 0.05-0.5% Sarkosyl, such as 0.1%, 0.2%, 0.3% OR 0.4% Sarkosyl. One exemplary washing buffer is 0.2% Sarkosyl in TBS.

The solid substrate, such as magnetic beads that have been contacted with the sample, can then be processed to detect bound prion protein, such as using a Standard QuIC(SQ) reaction or a real-time QuIC(RTQ) reaction, as discussed below. In one embodiment, prion proteins (e.g. PrP^{Sc}) bound to the antibody are not released (eluted) prior to detecting the bound prion proteins, rather the reaction mix including both the solid substrate comprising the antibody and the prion proteins are directly used in an assay to detect PrP-res or PrP^{Sc}, such as, but not limited to, SQ or RTQ. Thus, the immune complexes comprising the antibody that specifically binds PrP-res are not separated from the reaction mixture, but used directly in a SQ or RTQ assay.

III. QuIC(SQ) and RT-QuIC(RTQ)

The prion detection method termed protein misfolding cyclic amplification (PMCA) is based on the ability of prions to replicate in vitro in tissue homogenates containing PrP^{C} (see, for instance, PCT Publication No. WO0204954). PMCA involves amplification of PrP-res through incubation with a suitable prion protein substrate derived from brain tissue, serial amplification of the PrP, for instance by alternating incubation and sonication steps, and detection of the resulting PrP-res. In some instances, incubation and sonication are alternated over a period of approximately three weeks, and intermittently a portion of the reaction mix is removed and incubated with additional PrP^{C} in order to serially amplify the PrP-res in the sample. Following the repeated incubation/sonication/dilution steps, the resulting PrP-res is detected in the reaction mix. Although brain extract-based PMCA is a very sensitive assay for detecting PrP-res, it has a number of limitations, notably the time required to achieve optimal sensitivity (2-3 weeks) and the use of brain-derived PrP^{C} as the amplification substrate. This method also uses sonication.

In contrast, in the QuIC methods (SQ and RTQ), agitation is performed by shaking and not by sonication. These assays use recombinantly-expressed rPrP^{C} as a substrate (Atarashi et al., (2008) Nat Methods, 5, 211-212, incorporated herein by reference), which can be obtained rapidly in high purity and in large amounts, whereas purification of naturally occurring PrP^{C} from brain tissue is difficult and gives much lower yields (Deleault et al. (2005) J. Biol. Chem. 280, 26873-26879; Pan et al. (1993) Proc. Natl. Acad. Sci. USA 90, 10962-10966; Hornemann et al., (2004) EMBO Rep. 5, 1159-1164). Furthermore, unlike PrP^{C} in brain homogenates or purified from brain, rPrP^{C} can be easily mutated or strategically labeled with probes to simplify and accelerate the detection of relevant products.

There are two types of PrP-res amplification methods that utilize rPrP^{C}, one that uses sonication (rPrP-PMCA) (Atarashi et al., (2007) Nat Methods, 4, 645-650) and one that utilizes shaking (QuIC) (Atarashi et al., (2008) Nat Methods, 5, 211-212). These methods facilitate fundamental studies of the structure and conversion mechanism of PrP-res. Site-directed mutations can allow precise labeling of rPrP^{C} with a variety of probes that can report on conformational changes, and both inter-molecular and intra-molecular distances within rPrP-res^{(Sc)} aggregates. Furthermore, RTQ allows detection of the amplification product using thioflavin T (ThT). In enhanced RTQ, rPrP^{C} is preemptively replenished before much detectable (ThT-positive) polymerization has occurred (such as before 24 hours of incubation), while retaining the existing rPrP-res^{(Sc)}.

The QuIC and RT-QuIC methods generally involve mixing a sample (for example a tissue sample, CSF sample, or plasma sample that is suspected of containing prions or PrP-res) with purified rPrP^{C} to make a reaction mix, and performing a primary reaction to form and amplify specific forms of rPrP-res^{(Sc)} in the mixture. This primary reaction includes incubating the reaction mix to permit the PrP-res to initiate the conversion of rPrP^{C} to specific aggregates or polymers of rPrP-res^{(Sc)}; fragmenting any aggregates or polymers formed during the incubation step; and repeating the incubation and fragmentation steps one or more times, for instance from about 1 to 2 times, 1 to 4 times, 1 to 10 times, or 10 to about 50 times. In some embodiments of the method, serial amplification is carried out by removing a portion of the reaction mix and incubating it with additional rPrP^{C}. In other embodiments, additional rPrP^{C} is added to the reaction, such as during the lag phase (prior to the formation of detectable rPrP-res^{(Sc)}, such as prior to 24 hours of the reaction), and the incubation and fragmentation steps are repeated.

In further embodiments, the method is performed without serial amplification, such that substrate bound prions are retained in a reaction vessel, and that substrate is replenished without removing potential PrP-res seeds. For example, PrP-res can be amplified in a sample, by mixing the sample with purified rPrP^{C} to make a reaction mix; performing an amplification reaction that includes (i) incubating the reaction mix to permit coaggregation of the rPrP^{C} with the PrP-res/PrP^{Sc} that may be present in the reaction mix, and maintaining incubation conditions that promote coaggregation of the rPrP^{C} with the PrP-res and results in a conversion of the rPrP^{C} to rPrP-res^{(Sc)} while inhibiting development of rPrP-res^{(spon)}; (ii) agitating aggregates formed during step (i); (iii) optionally repeating steps (i) and (ii) one or more times. rPrP-res^{(Sc)} is detected in the reaction mix, wherein detection of rPrP-res^{(Sc)} in the reaction mix indicates that PrP-res was present in the sample.

Additional substrate (rPrP^{C}) can be added during the reaction, such as during the lag phase between the addition of the sample and the detection of rPrP-res^{(Sc)} formation. However, when a single round of amplification is used, a portion of the reaction mix is not removed and incubated with additional rPrP^{C}. In some embodiments, the rPrP^{C} can be replenished by adding additional rPrP^{C} substrate to the reaction mix.

Generally, with either QuIC or RT-QuIC (SQ or RTQ), the reaction includes the use of shaking in the absence of sonication (the QuIC reaction), and the use of cycles of shaking/rest that are about 1:1 in duration. In one non-limiting example, the reaction alternates 60 seconds of shaking and 60 seconds of no shaking (rest). In another non-limiting example, the reaction alternates 30 seconds of shaking and 30 seconds of no shaking (rest). However, the times can be varied, such as 45 seconds of shaking and 45 seconds of no shaking or 70 seconds of shaking and 70 seconds of no shaking. Thus the period of rest and the period of shaking are equal. In other embodiments, the period of rest and the period of shaking are about 120 seconds in length for the total cycle. Thus, in some examples, the reaction includes or 90 seconds of shaking and 30 seconds of no shaking, or 100 seconds of shaking and 20 seconds of no shaking, or 80 seconds of shaking and 40 seconds of rest. In additional embodiments, the total cycle time is about 60, 70, 80, 90, 100, 110 or 120 seconds in length and includes at least 30 seconds, at least 40, or at least 50 seconds of shaking.

Reactions have also been found to be particularly efficient at 37-60° C., for example 45-55° C., such as about 50° C., or at about 42° C. to 46° C. These conditions are particularly effective at promoting the formation of rPrP-res^{(Sc)} (notably the 17 kDa PK-resistant species), while reducing rPrP-res^{(spon)} formation within the first 24 hours of unseeded reactions. Thus, the reaction can be performed for 3 to 12 hours, such as 6 to 12 hours, such as 8 to 10 hours. However, longer amplification reactions of 14 hours, 16 hours, 20 hours, 24 hours, such as at least 45 hours, 48 hours or even 65 or 96 hours, can also provide excellent results, depending on the reaction temperature. In some embodiments, the reaction is performed for 3 to 96 hours. For example, the reaction can be performed for no more than 12 hours, no more than 24 hours, no more than 36 hours, no more than 48 hours, no more than 72 hours, no more than 96 hours or no more than 120 hours. In some examples the reaction is performed from about 5 hours to about 120 hours.

In some embodiments, the reaction is performed using sodium chloride (NaCl) at a concentration of 100 mM to 500 mM, such as about 100 mM, 200 mM, 300 mM, 400 mM NaCl. In other embodiments, the reaction is performed using 200 to 400 mM NaCl.

In methods wherein immunoprecipitation and real time QuIC is used (IP-RTQ reactions or eQuIC), ThT is used to detect rPrP-res^{(Sc)}. If the solid substrate is a bead, such as magnetic beads, the beads and any associated prions or prion-induced RTQ conversion products tend to cling to the bottom of reaction vessel, such as a well. Thus, the reaction fluid can easily be changed, and the substrate replenished in its pre-RTQ state, without removing many beads or bound reaction products from the well. The rPrP^{C} substrate can be replenished preemptively during the lag phase, such as before ThT positivity indicated much consumption by conversion to prion-seeded amyloid product. With replenishment, IP-RTQ is highly sensitive, such that the overall sensitivity of the RTQ was increased by at least 1000-fold and overall reaction time is greatly reduced. The concentration of substrate is generally 0.1 mg/ml.

Thus, QuIC reaction can be an RT-QuIC reaction, and thus can include thioflavin T (ThT) which allows detection of the rPrP-res^{(Sc)}. The RT-QuIC assay incorporates rPrP^{C} as a substrate, intermittent shaking of the reactions such as in 96-well plates, detergent- and chaotrope-free reaction conditions and ThT-based fluorescence detection of prion-seeded rPrP^{C} amyloid fibrils. One advantage of using ThT is that it can be included in the reaction mixture. Thioflavin T is a benzothiazole dye that exhibits enhanced fluorescence upon binding to amyloid fibrils (see Khurana et al., J. Structural Biol. 151: 229-238, 2005), and is commonly used to detect amyloid fibrils.

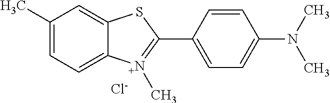

Following amplification, the prion-initiated rPrP-res^{(Sc)} in the reaction mix is detected. If ThT is included in the reaction (RT-QuIC), then rPrP-res^{(Sc)} can be detected using fluorescence at 450+/−10 nM excitation and 480+/−10 nm emission (see for example, Wilham et al., PLOS Pathogens 6(12): 1-15, 2010, incorporated herein by reference.) ThT can be included directly in the amplification mixture. In some embodiments, if ThT is included, the reaction mix does not include chaotropes or detergents. In some embodiments, if ThT is included, the reaction mix does not include chaotropic agents or detergents that can alter the rPrP-res^{(Sc)}-sensitivity of ThT. In one non-limiting example, when 15B3 immunoprecipitation is used with RTQ reactions the final concentration of ThT in each reaction is 1 mM. In other examples, ThT is used at a final concentration of about 0.1 to 1 mM in the reaction.

The sodium chloride (NaCl) concentration can be varied in the reaction. In some embodiments, a concentration of about 200-400 mM NaCl allows sensitive detection of hamster, sheep, deer and human PrP-res while reducing the incidence of spontaneous conversion of the substrate. In some embodiments, detergent at a concentration of greater than 0.002% is not included in an RTQ reaction.

If QuIC is utilized, PrP-res can be detected by means other than ThT fluorescence, for example, using an antibody (see below). In some examples, the reaction mix is digested with proteinase K (which digests the remaining rPrP^{C} in the reaction mix) prior to detection of the rPrP-res^{(Sc)}. Two types of mis-folded prion protein can be generated in QuIC reactions, one occurring spontaneously (rPrP-res^{(spon)}) and the other initiated by the presence of prions (rPrP-res^{(Sc)}) in the test sample. Thus, discrimination between the former and the latter can be done on the basis of differing protein fragment sizes generated upon exposure to proteinase K. An unexpectedly superior decrease in the amount of rPrP-res^{(spon)} formed is achieved with the QuIC assays. Thus, RT-QuIC(RTQ) (which includes thioflavin T) reactions need not be subjected to proteinase K treatment. Thus, this step is optional.

All of the methods disclosed herein, such as SQ and RTQ, will work under a variety of conditions. In several embodiments, optimal conditions that support specific prion/PrP-res-seeded SQ include the use of a detergent, such as an ionic and/or a non-ionic detergent. The conditions can include the use of about 0.05-0.1% of an ionic detergent, such as SDS. The conditions also can include the use of about 0.05-0.1% of a nonionic detergent such as TX-100 in the reaction mixture.

With regard to the PrP^{C} substrate, it has also been found that the SQ and RTQ assays can perform cross-species amplification of target PrP-res. In fact, rHaPrP and chimeric rPrP^{C} can be used in SQ and RTQ reactions for amplification of human prions. rHaPrP appears to have a structure that promotes the formation of these aggregates with minimal formation of rPrP-res^{(spon)} byproduct. Hence rHaPrP^{C}, and chimeras including HaPrP components, can be used to amplify target PrP-res in a sample taken from a species other than a hamster, such as a sample taken from a human, sheep, cow or cervid.

The PrP^{C} in used in the reaction can be recombinant prion protein, for example prion protein from cells engineered to over express the protein. Any prion protein sequence can be used to generate the rPrP^{C}, for instance: Xenopus laevis (GENBANK® Accession No: NP001082180), Bos Taurus (GENBANK® Accession No: CAA39368), Danio verio (GENBANK® Accession No: NP991149), Tragelaphus strepsiceros (GENBANK® Accession No: CAA52781), Ovis aries (GENBANK® Accession No: CAA04236), Trachemys scripta (GENBANK® Accession No: CAB81568), Gallus gallus (GENBANK® Accession No: AAC28970), Rattus norvegicus NP036763), Mus musculus (GENBANK® Accession No: NP035300), Monodelphis domestica (GENBANK®Accession No: NP001035117), Homo sapiens (GENBANK® Accession No: BAA00011), Giraffa camelopardalis (GENBANK® Accession No: AAD13290), Oryctolagus cuniculus (GENBANK® Accession No: NP001075490), Macaca mulatta (GENBANK® Accession No: NP001040617), Bubalus bubalus (GENBANK® Accession No: AAV30514), Tragelaphus imberbis (GENBANK®Accession No: AAV30511), Boselaphus tragocamelus (GENBANK® Accession No: AAV30507), Bos gams (GENBANK® Accession No: AAV 30505), Bison bison (GENBANK® Accession No: AAV30503), Bos javanicus (GENBANK® Accession No: AAV30498), Syncerus coffer coffer (GENBANK® Accession No: AAV30492), Syncerus coffer nanus (GENBANK® Accession No: AAV30491), and Bos indicus (GENBANK® Accession No: AAV30489). These GENBANK® sequences are incorporated herein by reference.

In some embodiments, only a partial prion protein sequence is expressed as rPrP^{C}. For instance, in certain examples rPrP^{C} includes amino acids 23-231 (SEQ ID NOS: 1, 2) of the hamster (SEQ ID NO: 8) or mouse (SEQ ID NO: 9) prion protein sequences, or the corresponding amino acids of other prion protein sequences, for instance amino acids 23-231 (SEQ ID NO: 3) of human (129M) prion protein (SEQ ID NO: 10), amino acids 23-231 (SEQ ID NO: 4) of human (129V) prion protein (SEQ ID NO: 11), amino acids 25-241 (SEQ ID NO: 5) of bovine (6-octarepeat) prion protein, amino acids 25-233 (SEQ ID NO: 6) of ovine (136A 154R 171Q) prion protein, or amino acids 25-234 (SEQ ID NO: 7) of deer (96G 132M 138S) prion protein. The partial prion protein sequence expressed as rPrP^{C} can correspond to the polypeptide sequences of the natural mature full-length PrP^{C} molecule, meaning that the rPrP^{C} polypeptide lacks both the amino-terminal signal sequence and carboxy-terminal glycophosphatidylinositol-anchor attachment sequence. In another example, amino acids 30-231, 40-231, 50-231, 60-231, 70-231, 80-231, or 90-231 of any one of human, human 129V, bovine, ovine, or deer are utilized in the assays described herein. One of skill in the art can readily produce these polypeptides using the sequence information provided in SEQ ID NOs: 1-11, or using information available in GENBANK® (as available on Jul. 20, 2007).

The rPrP^{C} can be a chimeric rPrP^{C}, wherein a portion of the protein is from one species, and a portion of the protein is from another species, can also be utilized. In one example about 10 to about 90%, such as about 10%, about 20%, about 30%, about 40%, about 50%, about 60%, about 70% about 80% or about 90% of the rPrP^{C} is from one species, and, correspondingly, about 90%, about 80%, about 70%, about 60%, about 50%, about 40%, about 30%, about 20% or about 10% is from another species. Chimeric proteins can include, for example, hamster rPrP^{C} and rPrP^{C} from another species, such as human PrP. In another embodiment, the chimeric protein includes hamster PrP^{C} and sheep PrP. A chimeric hamster-sheep rPrP^{C} construct can be used, such as, but not limited to, for the detection of human vCJD prions. In one embodiment, a chimeric rPrP^{C} (designated Ha-S PrP) is used, wherein the chimeric molecule includes residues 23-137 were of the Syrian hamster sequence and the remaining residues 138-231 were homologous to sheep residues 141-234 (R154,Q171 polymorph).

In some embodiments, to produce rPrP^{C}, host cells are transformed with a nucleic acid vector that expresses the rPrP^{C}, for example human, cow, sheep or hamster rPrP^{C}, or a chimeric form thereof. These cells can be mammalian cells, bacterial cells, yeast cells, insect cells, or whole organisms, such as transgenic mice. Other cells also can serve as sources of the PrP. In particular examples the cell is a bacterial cell, such as an E. coli cell. Purified rPrP^{C} from rPrP^{C} expressing cells or, in some cases, raw cell lysates, can be used as the source of the non-pathogenic protein.

In some embodiments the recombinant protein is fused with an additional amino acid sequence. For example, over expressed protein can be tagged for purification or to facilitate detection of the protein in a sample. Some possible fusion proteins that can be generated include histidine tags, Glutathione S-transferase (GST), Maltose binding protein (MBP), green fluorescent protein (GFP), and Flag and myc-tagged rPrP. These additional sequences can be used to aid in purification and/or detection of the recombinant protein, and in some cases are subsequently removed by protease cleavage. For example, coding sequence for a specific protease cleavage site can be inserted between the PrP^{C} coding sequence and the purification tag coding sequence. One example for such a sequence is the cleavage site for thrombin. Thus, fusion proteins can be cleaved with the protease to free the PrP^{C} from the purification tag.

Any of the wide variety of vectors known to those of skill in the art can be used to over-express rPrP^{C}. For example, plasmids or viral vectors can be used. These vectors can be introduced into cells by a variety of methods including, but not limited to, transfection (for instance, by liposome, calcium phosphate, electroporation, particle bombardment, and the like), transformation, and viral transduction.

Recombinant PrP^{C} also can include proteins that have amino sequences containing substitutions, insertions, deletions, and stop codons as compared to wild type sequences. In certain embodiments, a protease cleavage sequence is added to allow inactivation of protein after it is converted into prion form. For example, cleavage sequences recognized by Thrombin, Tobacco Etch Virus (Life Technologies, Gaithersburg, Md.) or Factor Xa (New England Biolabs, Beverley, Mass.) proteases can be inserted into the sequence. In some embodiments, inactivation of protein after it is converted into the PrP-res seeded form is unnecessary because the rPrP-res^{(Sc)} resulting from the reaction has little or no infectivity.

Changes also can be made in the pPrP^{C} protein coding sequence, for example in the coding sequence for mouse, human, bovine, sheep, goat, deer and/or elk prion protein (GENBANK® accession numbers NM_{—}011170, NM_{—}183079, AY335912, AY723289, AY723292, AF156185 and AY748455, respectively, all of which are incorporated herein by reference, Jul. 20, 2007). For example, mutations can be made to match a variety of mutations and polymorphisms known for various mammalian prion protein genes (see, for instance, Table 1). Furthermore, chimeric PrP molecules comprising sequences from two or more different natural PrP sequences (for instance from different host species or strains) can be expressed from vectors with recombinant PrP gene sequences, and such chimeras can be used for RT-QuIC and QuIC detection of prion from various species. Cells expressing these altered prion protein genes can be used as a source of the rPrP^{C}, and these cells can include cells that endogenously express the mutant rPrP gene, or cells that have been made to express a mutant rPrP protein by the introduction of an expression vector. Use of a mutated rPrP^{C} can be advantageous, because some of these proteins are more easily or specifically converted to protease-resistant forms, or are less prone to spontaneous (prion-independent) conversion, and thus can further enhance the sensitivity of the method.

In certain embodiments, cysteine residues are placed at positions 94 and 95 of the hamster prion protein sequence in order to be able to selectively label the rPrP at those sites using sulfhydryl-reactive labels, such as pyrene and fluorescein linked to maleimide-based functional groups. In certain embodiments, these tags do not interfere with conversion but allow much more rapid, fluorescence-based detection of the reaction product. In one example, pyrenes in adjacent molecules of rPrP-res^{(Sc)} are held in close enough proximity to allow eximer formation, which shifts the fluorescence emission spectrum in a distinct and detectable manner. Free pyrenes released from, or on, unconverted rPrP^{C} molecules are unlikely to form eximer pairs. Thus, the reaction can be run in a multiwell plate, digested with proteinase K, and then eximer fluorescence can be measured to rapidly test for the presence of rPrP-res^{(Sc)}. Sites 94 and 95 were chosen for the labels because the PK-resistance in this region of PrP-res distinguishes rPrP-res^{(Sc)} from rPrP-res^{(spon)}, giving rise to the 17 kDa rPrP-res^{(Sc)} band. Other positions in the PK-resistant region(s) that distinguish the 17-kDa rHa PrP-res^{(Sc)} fragment from all rHaPrP-res^{(spon)} fragments also can work for this purpose.

TABLE 1

| | Human | Ovine | Bovine |
|---|---|---|---|
| Pathogenic human | polymor- | polymor- | polymor- |
| mutations | phisms | phisms | phisms |
| 2 octarepeat insert | Codon 129 | Codon 171 | 5 or 6 |
| | Met/Val | Arg/Glu | octarepeats |
| 4-9 octarepeat insert | Codon 219 | Codon 136 | |
| | Glu/Lys | Ala/Val | |
| Codon 102 Pro-Leu | | | |
| Codon 105 Pro-Leu | | | |
| Codon 117 Ala-Val | | | |
| Codon 145 Stop | | | |
| Codon 178 Asp-A | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

Recombinant prion proteins (rPrP^{C}) can be produced by any methods known to those of skill in the art. In one example, in vitro nucleic acid amplification (such as polymerase chain reaction (PCR)) can be utilized as a method for producing nucleic acid sequences encoding prion proteins. PCR is a standard technique that is described, for instance, in PCR Protocols: A Guide to Methods and Applications (Innis et al., San Diego, Calif.: Academic Press, 1990), or PCR Protocols, Second Edition (Methods in Molecular Biology, Vol. 22, ed. by Bartlett and Stirling, Humana Press, 2003).

A representative technique for producing a nucleic acid sequence encoding a recombinant prion protein by PCR involves preparing a sample containing a target nucleic acid molecule that includes the prion protein-encoding sequence. For example, DNA or RNA (such as mRNA or total RNA) can serve as a suitable target nucleic acid molecule for PCR reactions. Optionally, the target nucleic acid molecule can be extracted from cells by any one of a variety of methods well known to those of ordinary skill in the art (for instance, Sambrook et al., Molecular Cloning: A Laboratory Manual, Cold Spring Harbor Laboratory Press, New York, 1989; Ausubel et al., Current Protocols in Molecular Biology, Greene Publ. Assoc. and Wiley-Intersciences, 1992). Prion proteins are expressed in a variety of mammalian cells. In examples where RNA is the initial target, the RNA is reverse transcribed (using one of a myriad of reverse transcriptases commonly known in the art) to produce a double-stranded template molecule for subsequent amplification. This particular method is known as reverse transcriptase (RT)-PCR. Representative methods and conditions for RT-PCR are described, for example, in Kawasaki et al. (In PCR Protocols, A Guide to Methods and Applications, Innis et al. (eds.), 21-27, Academic Press, Inc., San Diego, Calif., 1990).

The selection of amplification primers will be made according to the portion(s) of the target nucleic acid molecule that is to be amplified. In various embodiments, primers (typically, at least 10 consecutive nucleotides of prion-encoding nucleic acid sequence) can be chosen to amplify all or part of a prion-encoding sequence. Variations in amplification conditions may be required to accommodate primers and amplicons of differing lengths and composition; such considerations are well known in the art and are discussed for instance in Innis et al. (PCR Protocols, A Guide to Methods and Applications, San Diego, Calif.: Academic Press, 1990). From a provided prion protein-encoding nucleic acid sequence, one skilled in the art can easily design many different primers that can successfully amplify all or part of a prion protein-encoding sequence.

As described herein, a number of prion protein-encoding nucleic acid sequences are known. Though particular nucleic acid sequences are disclosed, one of skill in the art will appreciate that also provided are many related sequences with the functions described herein, for instance, nucleic acid molecules encoding conservative variants of a prion protein. One indication that two nucleic acid molecules are closely related (for instance, are variants of one another) is sequence identity, a measure of similarity between two nucleic acid sequences or between two amino acid sequences expressed in terms of the level of sequence identity shared between the sequences. Sequence identity is typically expressed in terms of percentage identity; the higher the percentage, the more similar the two sequences.

Methods for aligning sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; Huang, et al., Computer Applications in the Biosciences 8:155-165, 1992; Pearson et al., Methods in Molecular Biology 24:307-331, 1994; Tatiana et al., (1999), FEMS Microbiol. Lett., 174:247-250, 1999. Altschul et al. present a detailed consideration of sequence-alignment methods and homology calculations (J. Mol. Biol. 215:403-410, 1990).

The National Center for Biotechnology Information (NCBI) Basic Local Alignment Search Tool (BLAST™, Altschul et al., J. Mol. Biol. 215:403-410, 1990) is available from several sources, including the National Center for Biotechnology Information (NCBI, Bethesda, Md.) and on the Internet, for use in connection with the sequence-analysis programs blastp, blastn, blastx, tblastn and tblastx. A description of how to determine sequence identity using this program is available on the internet under the help section for BLAST™.

For comparisons of amino acid sequences of greater than about 30 amino acids, the “Blast 2 sequences” function of the BLAST™ (Blastp) program is employed using the default BLOSUM62 matrix set to default parameters (cost to open a gap [default=5]; cost to extend a gap [default=2]; penalty for a mismatch [default=−3]; reward for a match [default=1]; expectation value (E) [default=10.0]; word size [default=3]; number of one-line descriptions (V) [default=100]; number of alignments to show (B) [default=100]). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 50%, at least 60%, at least 70%, at least 80%, at least 85%, at least 90%, at least 95%, at least 98%, or at least 99% sequence identity to the sequence of interest.

For comparisons of nucleic acid sequences, the “Blast 2 sequences” function of the BLAST™ (Blastn) program is employed using the default BLOSUM62 matrix set to default parameters (cost to open a gap [default=11]; cost to extend a gap [default=1]; expectation value (E) [default=10.0]; word size [default=11]; number of one-line descriptions (V) [default=100]; number of alignments to show (B) [default=100]). Nucleic acid sequences with even greater similarity to the reference sequences will show increasing percentage identities when assessed by this method, such as at least 60%, at least 70%, at least 75%, at least 80%, at least 85%, at least 90%, at least 95%, or at least 98%, or at least 99% sequence identity to the prion sequence of interest.

Another indication of sequence identity is nucleic acid hybridization. In certain embodiments, prion protein-encoding nucleic acid variants hybridize to a disclosed (or otherwise known) prion protein-encoding nucleic acid sequence, for example, under low stringency, high stringency, or very high stringency conditions. Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, although wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following are representative hybridization conditions and are not meant to be limiting.

Very High Stringency (Detects Sequences that Share at Least 90% Sequence Identity)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Detects Sequences that Share at Least 80% Sequence Identity)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Detects Sequences that Share at Least 50% Sequence Identity)
Hybridization: 6×SSC at RT to 55° C. for 16-20 hours
Wash at least twice: 2×-3×SSC at RT to 55° C. for 20-30 minutes each.

Prion protein variants, that include the substitution of one or several amino acids for amino acids having similar biochemical properties (so-called conservative substitutions), can also be used in the presently described methods. Conservative amino acid substitutions are likely to have minimal impact on the activity of the resultant protein, such as its ability to convert to PrP-res. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. In some examples, prion protein variants can have no more than 3, 5, 10, 15, 20, 25, 30, 40, or 50 conservative amino acid changes. The following table shows exemplary conservative amino acid substitutions that can be made to a prion protein, for instance the recombinant prion proteins shown in SEQ ID NOs: 1-7, such that thy can still be used in the presently claimed assays.

TABLE 2

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

To purify PrP^{C} from recombinant (or natural) sources, the composition is subjected to fractionation to remove various other components from the composition. Various techniques suitable for use in protein purification are well known. These include, for example, precipitation with ammonium sulfate, PTA, PEG, antibodies and the like, or by heat denaturation followed by centrifugation; chromatography steps such as metal chelate, ion exchange, gel filtration, reverse phase, hydroxylapatite, lectin affinity, and other affinity chromatography steps; isoelectric focusing; gel electrophoresis; and combinations of such and other techniques.

IV. Sources of Samples for rPrP-res^{(Sc)} Amplification Assays

The samples analyzed using the methods described herein can include any composition capable of being contaminated with a prion. Such compositions can include tissue samples, biopsy samples, or bodily fluids including, but not limited to, plasma, blood, lymph nodes, brain, spinal cord, tonsils, spleen, skin, muscles, appendix, olfactory epithelium, cerebral spinal fluid, urine, feces, milk, intestines, tears and/or saliva. The sample can be a human sample or a veterinary sample, such as, but not limited to a sample from a cow, sheep or deer. The presently disclosed methods also can be used for prion-free herd/flock certification, such as in cattle, sheep, and cervids.

Other compositions from which samples can be taken for analysis, for instance, include food stuffs, pharmaceutical agents (such as animal-derived biological agents), drinking water, forensic evidence, surgical implements, and/or mechanical devices. Thus, samples that can be tested include biotechnology products and environmental samples (such as water, soils, plants, landfills, sewage) and agriculture samples (such as animal-based foods, animal-based feeds and nutritional supplements, animal waste products, byproducts, carcasses, slaughterhouse wastes, specified risk materials) to ensure there is no contamination by prions.

V. Methods for Detecting rPrP-res^{(Sc)} in Amplification Mixes in the Absence of ThT

Once rPrP-res^{(Sc)} has been generated using rPrP^{C} amplification, such as using rPrP-PMCA (such as the QUIC assay), rPrP-res^{(Sc)} can be detected in the reaction mixture. Direct and indirect methods can be used for detection of rPrP-res^{(Sc)} in a reaction mixture. Detection using ThT is described above. For methods in which rPrP-res^{(Sc)} is directly detected, separation of newly-formed rPrP-res^{(Sc)} from remaining rPrP^{C} usually is required. This typically is accomplished based on the different natures of rPrP-res^{(Sc)} versus rPrP^{C}. For instance, rPrP-res^{(Sc)} typically is highly insoluble and resistant to protease treatment. Therefore, in the case of rPrP-res^{(Sc)} and rPrP^{C}, separation can be by, for instance, protease treatment. Lateral flow assays or SOPHIA can also be used.

A. Protease Treatment

When rPrP-res^{(Sc)} and rPrP^{C} are separated by protease treatment, reaction mixtures are incubated with, for example, Proteinase K (PK). An exemplary protease treatment includes digestion of the protein, for instance, rPrP^{C}, in the reaction mixture with 1-20 μg/ml of PK for about 1 hour at 37° C. Reactions with PK can be stopped prior to assessment of prion levels by addition of PMSF or electrophoresis sample buffer. Depending on the nature of the sample, incubation at 37° C. with 1-50 μg/ml of PK generally is sufficient to remove rPrP^{C}.

rPrP-res^{(Sc)} also can be separated from the rPrP^{C} by the use of ligands that specifically bind and precipitate the misfolded form of the protein, including conformational antibodies, certain nucleic acids, plasminogen, PTA and/or various peptide fragments.

B. Western Blot

In some examples, reaction mixtures fractioned or treated with protease to remove rPrP^{C} are then subjected to Western blot for detection of rPrP-res^{(Sc)} and the discrimination of rPrP-res^{(Sc)} from rPrP-res^{(spon)}. Typical Western blot procedures begin with fractionating proteins by sodium dodecyl sulfate-polyacrylamide gel electrophoresis (SDS-PAGE) under reducing conditions. The proteins are then electroblotted onto a membrane, such as nitrocellulose or PVDF and probed, under conditions effective to allow immune complex (antigen/antibody) formation, with an anti-prion protein antibody. Exemplary antibodies for detection of prion protein include the 3F4 monoclonal antibody, monoclonal antibody D13 (directed against residues 96-106 (Peretz et al. (2001) Nature 412, 739-743)), polyclonal antibodies R18 (directed against residues 142-154), and R20 (directed against C-terminal residues 218-232) (Caughey et al. (1991) J. Virol. 65, 6597-6603).

Following complex formation, the membrane is washed to remove non-complexed material. An exemplary washing procedure includes washing with a solution such as PBS/Tween, or borate buffer. The immunoreactive bands are visualized by a variety of assays known to those in the art. For example, the enhanced chemoluminesence assay (Amersham, Piscataway, N.J.) can be used.

If desired, prion protein concentration can be estimated by Western blot followed by densitometric analysis, and comparison to Western blots of samples for which the concentration of prion protein is known. For example, this can be accomplished by scanning data into a computer followed by analysis with quantitation software. To obtain a reliable and robust quantification, several different dilutions of the sample generally are analyzed in the same gel.

C. ELISA, Immunochromatographic Strip Assay, and Conformation Dependent Immunoassay

As described above, immunoassays in their most simple and direct sense are binding assays. Specific non-limiting immunoassays of use include various types of enzyme linked immunosorbent assays (ELISAs), immunochromatographic strip assays, radioimmunoassays (RIA), and specifically conformation-dependent immunoassays.

In one exemplary ELISA, anti-PrP antibodies are immobilized onto a selected surface exhibiting protein affinity, such as a well in a polystyrene microtiter plate. Then, a reaction mixture suspected of containing prion protein antigen is added to the wells. After binding and washing to remove non-specifically bound immune complexes, the bound prion protein can be detected. Detection generally is achieved by the addition of another anti-PrP antibody that is linked to a detectable label. This type of ELISA is a simple “sandwich ELISA.” Detection also can be achieved by the addition of a second anti-PrP antibody, followed by the addition of a third antibody that has binding affinity for the second antibody, with the third antibody being linked to a detectable label.

In another exemplary ELISA, the reaction mixture suspected of containing the prion protein antigen is immobilized onto the well surface and then contacted with the anti-PrP antibodies. After binding and washing to remove non-specifically bound immune complexes, the bound anti-prion antibodies are detected. Where the initial anti-PrP antibodies are linked to a detectable label, the immune complexes can be detected directly. Again, the immune complexes can be detected using a second antibody that has binding affinity for the first anti-PrP antibody, with the second antibody being linked to a detectable label.

Another ELISA in which protein of the reaction mixture is immobilized involves the use of antibody competition in the detection. In this ELISA, labeled antibodies against prion protein are added to the wells, allowed to bind, and detected by means of their label. The amount of prion protein antigen in a given reaction mixture is then determined by mixing it with the labeled antibodies against prion before or during incubation with coated wells. The presence of prion protein in the sample acts to reduce the amount of antibody against prion available for binding to the well and thus reduces the ultimate signal. Thus, the amount of prion in the sample can be quantified.

Irrespective of the format employed, ELISAs have certain features in common, such as coating, incubating or binding, washing to remove non-specifically bound species, and detecting the bound immune complexes. These are described below.

In coating a plate with either antigen or antibody, one generally incubates the wells of the plate with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antibodies. These include bovine serum albumin, casein, and solutions of milk powder. The coating allows for blocking of nonspecific adsorption sites on the immobilizing surface, and thus reduces the background caused by nonspecific binding of antibodies onto the surface.

It is customary to use a secondary or tertiary detection means rather than a direct procedure with ELISAs, though this is not always the case. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin, milk proteins, and phosphate buffered saline (PBS)/Tween. These added agents also tend to assist in the reduction of nonspecific background. “Suitable” conditions also mean that the incubation is at a temperature and for a period of time sufficient to allow effective binding. Incubation steps are typically from about 1 to 2 to 4 hours, at temperatures preferably on the order of 25° C. to 27° C., or can be overnight at about 4° C. or so.

Following all incubation steps in an ELISA, the contacted surface is washed so as to remove non-complexed material. An exemplary washing procedure includes washing with a solution such as PBS/Tween or borate buffer. Following the formation of specific immune complexes between the test sample and the originally bound material, and subsequent washing, the occurrence of even minute amounts of immune complexes can be determined.

To provide a detecting means, the second or third antibody generally will have an associated label to allow detection. In some examples, this is an enzyme that will generate color development upon incubating with an appropriate chromogenic substrate. Thus, for example, the first or second immune complex is contacted and incubated with a urease, glucose oxidase, alkaline phosphatase or hydrogen peroxidase-conjugated antibody for a period of time and under conditions that favor the development of further immune complex formation (for instance, incubation for two hours at room temperature in a PBS-containing solution such as PBS-Tween).

After incubation with the labeled antibody, and subsequent to washing to remove unbound material, the amount of label is quantified, for instance, by incubation with a chromogenic substrate such as urea and bromocresol purple or 2,2′-azino-di-(3-ethyl-benzthiazoline-6-sulfonic acid) and H₂O₂, in the case of peroxidase as the enzyme label. Quantification is then achieved by measuring the degree of color generation, for instance, using a visible spectra spectrophotometer.

D. rPrP^{C} Labeling

In certain embodiments, the recombinant rPrP^{C} substrate protein can be labeled to enable high sensitivity of detection of protein that is converted into rPrP-res^{(Sc)}. For example, rPrP^{C} can be radioactively labeled, epitope tagged, or fluorescently labeled. The label can be detected directly or indirectly. Radioactive labels include, but are not limited to ¹²⁵I, ³²P, ³³P, and ³⁵S.

The mixture containing the labeled protein is subjected to an amplification assay, such as QuIC, and the product detected with high sensitivity by following conversion of the labeled protein after removal of the unconverted protein for example by proteolysis. Alternatively, the protein can be labeled in such a way that a signal can be detected upon the conformational changes induced during conversion. An example of this is the use of FRET technology, in which the protein is labeled by two appropriate fluorophores, which upon refolding become close enough to exchange fluorescence energy (see for example U.S. Pat. No. 6,855,503).

In certain embodiments, cysteine residues are placed at positions 94 and 95 of the hamster prion protein sequence in order to be able to selectively label the rPrP^{C} at those sites using sulfhydryl-reactive labels, such as pyrene and fluorescein linked to maleimide-based functional groups. In certain embodiments, these tags do not interfere with conversion but allow much more rapid, fluorescence-based detection of the reaction product. In one example, pyrenes in adjacent molecules of rPrP-res^{(Sc)} are held in close enough proximity to allow eximer formation, which shifts the fluorescence emission spectrum in a distinct and detectable manner. Free pyrenes released from, or on, unconverted rPrP^{C} molecules are unlikely to form eximer pairs. Thus, the rPrP-res^{(Sc)} amplification reaction can be run in a multiwell plate, digested with proteinase K, and then eximer fluorescence can be measured to rapidly test for the presence of rPrP-res^{(Sc)}. Sites 94 and 95 are chosen for the labels because the PK-resistance in this region of constituent PrP molecules distinguishes rPrP-res^{(Sc)} from rPrP-res^{(spon)}, giving rise to the 17 kDa rPrP-res^{(Sc)} band. Other positions in the PK-resistant region(s) that distinguish the 17-kDa rPrP-res^{(Sc)} fragment from all rPrP-res^{(spon)} fragments also can work for this purpose.

In certain other embodiments, the use of a fluorescently-tagged rPrP^{C} substrate for the reaction is combined with the use an immunochromatographic strip test with an immobilized rPrP-res^{(Sc)} specific antibody (for example, from Prionics AG, Schlieren-Zurich, Switzerland). Binding of the rPrP-res^{(Sc)} to the antibody is then detected with a fluorescence detector.

The disclosure is illustrated by the following non-limiting Examples.

### EXAMPLES

TSEs are largely untreatable and difficult to diagnose definitively prior to irreversible clinical decline or death. The transmissibility of TSEs within and between species highlights the need for practical tests for even the smallest amounts of infectivity. Currently, most in vitro methods have major limitations that would preclude their use in routine diagnostic or screening applications.

A key challenge in managing transmissible spongiform encephalopathies (TSEs) or prion diseases in medicine, agriculture, and wildlife biology is the development of practical tests for prions that are at, or below, infectious levels. Of particular interest are tests capable of detecting prions in blood components such as plasma, but blood typically has extremely low prion concentrations and contains inhibitors of the most sensitive prion tests. As disclosed herein, coupling of immunoprecipitation and an improved real time QuIC reaction dramatically enhanced detection of variant Creutzfeldt-Jakob disease (vCJD) brain tissue diluted into human plasma. Dilutions of 10¹⁴-fold, containing ˜2 ag/ml of proteinase K-resistant prion protein, were readily detected, indicating ˜10,000-fold greater sensitivity for vCJD brain than has previously been reported. Plasma and serum samples from scrapie-infected and uninfected hamsters were discriminated, even in early preclinical stages. This combined assay, termed enhanced QuIC (eQuIC), provides a markedly sensitive assay that can be used routine detection of low levels of prions in tissues, fluids or environmental samples.

### Example 1

### Exemplary Methods

Provided below are exemplary methods for detecting prion proteins that include immunoprecipitation and amplification. The protocols provided should not be construed to be limiting.

### A) 15B3 Coating of DYNABEADS®

1. Vortex Rat anti-Mouse IgM DYNABEADS® (Invitrogen, cat. No. 110.39D) for 30 seconds.
2. Transfer an aliquot of beads (e.g. 250 μl) to new tube for 15B3 antibody coating procedure.
3. Place tube on magnet for 2 minutes and remove bead storage buffer.
4. Add fresh Coating Buffer (0.1% BSA in 1×PBS, filtered and kept at −4° C.):
5-fold the original bead volume (e.g. 1250 μl).
5. Vortex.
6. Place tube on magnet for 2 minutes and remove Coating Buffer (see above).
7. Repeat steps 4 & 5.
8. Add 5-fold the original bead volume (e.g. 1250 μl) of Coating Buffer.
9. Add 15B3 antibody to resuspended beads at a final concentration of 360 μg/ml.
10. Incubate with “end-over-end” rotation at room temperature for 2 hours.
11. Wash three times with 5-fold the initial bead volume of Coating Buffer.
12. Resuspend beads in original volume with Coating Buffer (e.g. 250 μl) & store at −4° C.

### B) 15B3 Immunoprecipitation of 263K/vCJD PrP-res in 500 μl of Human Plasma

1. Vortex 15B3 coated beads for 30 seconds.
2. Aliquot 40 μl of vortexed 15B3-coated beads per tube/sample.
3. Place tubes containing 15B3-coated beads on magnet for 2 minutes and remove Coating buffer.
4. Add 500 μl of Immunoprecipitation Buffer (0.4% Sarkosyl in 1×TBS) to beads.
1. Add 500 μl of plasma to the beads in Immunoprecipitation Buffer (total volume in tube will be 1 ml).
2. Incubate at 37 C.° for 24 hours with “end-over-end” rotation.
3. Place tubes on magnet 2 minutes and eliminate buffer.
4. Wash twice with 500 μl per tube of 0.2% Sarkosyl in 1×TBS (Tris buffered saline) Buffer.
5. Resuspend washed beads in 10 μl of 1×PBS (phosphate buffered saline, filtered and kept at room temperature) and use 2 μl to seed Standard- or Real Time-QuIC

### C) Standard QuIC(SQ) Reaction

### Materials:

a. Reaction tubes: 0.5 ml conical microcentrifuge tubes with screw caps (Fisher 02-681-334)
b. 15B3-coated beads in 1×PBS used to immunoprecipitate prion seeding activity in plasma and kept at 4° C.
c. Hamster 23-231 rPrP^{C} in 10 mM Sodium Phosphate Buffer (pH5.8)
d. 4×QUIC buffer (Final composition: 0.4% SDS, 0.4% TritonX-100, and 4×PBS):

| | |
|---|---|
| 10% SDS stock | (40 μl/ml) |
| 10% TritonX-100 stock | (40 μl/ml) |

10×PBS stock (400 μl/ml), pH 6.9:

| | | |
|---|---|---|
| Na₂HPO₄7H₂0 | 26.8 | g/L |
| NaH₂PO₄H₂0 | 13.8 | g/L |
| NaCl | 75.9 | g/L |
| MilliQ H₂O | (520 | μl/ml) |

### Protocol:

1) Thaw hamster (23-231) rPrP^{C} and filter with a 100 kD microtube filter (PALL) by spinning 500 μl of protein at 4000×g for 5 min.
2) Dilute rPrP^{C} 1:10 in 0.1% SDS/PBS and measure UV absorbance at 280 nm: [Protein mg/mL]=[280 nm absorbance/2.6 (i.e., the PrP extinction coefficient)×Dilution Factor=X mg/mL, wherein X=rPrP^{C} stock concentration; Ideal protein concentration will be between 0.4 and 0.3 mg/ml.
   Note:
      Want 0.1 mg/mL rPrP^{C} in 100 μL reaction=10 μg/X=Y μL rPrP^{C} per reaction (where Y=volume of rPrP^{C} to be added to achieve a final concentration of 0.1 mg/ml per reaction)
      X and Y are variables: X is the concentration of the rPrP^{C} which can vary depending on the specific preparation, and Y is the volume of protein added to the reaction to have a final concentration of 0.1 mg/ml
      Amount of water in reaction=100−Y−2−25=Z μL Water per reaction (where Z=volume of water that will be added to achieve a final reaction volume of 100 μL). The final reaction volume is 100 μL, so once the volume of rPrPC is established that needs to be added along with all the other components (e.g. NaCl, PBS), the remaining volume to get to 100 ul is water.
3) Prepare reaction mixture in tubes as described above (added in the order specified).

### 1^{st} Round Reaction Mixture:

| Z | μl | MilliQ H₂O |
|---|---|---|
| 25 | μl | 4X QUIC buffer |
| 2 | μl | 15B3 beads in 1XPBS |
| Y | μl | rPrP^{-Sen} |
| 100 | μl | total volume |

Wherein Z = 73 − Y

Vortex first three components for 5 s prior to adding the rPrP^{C}. Add the rPrP^{C} gently, to avoid creation of bubbles. Cap reaction tubes, but do not vortex.
4) Place tubes in Eppendorf THERMOMIXER® with 24×0.5 ml tube block.
5) Incubate tubes in THERMOMIXER® R for 8-10 hours at 50° C., alternating between 60 seconds of shaking at 1500 rpm and no shaking for 60 sec.
6) Spin the tubes briefly to bring any solution down out of the caps.
7) Remove aliquot for 2^{nd} QuIC round and/or prepare for PK digestion and immunoblot analysis (see below).

### 2^{nd} Standard QuIC Round:

1) Prepare reaction mixture in fresh reaction tubes similar to 1^{st} round described above. Note: gently vortex sample tubes to evenly suspend any pellet just prior to transferring volume to seed the 2^{nd} round reaction tube.
2) Filter and measure A280 of rPrP^{C} as stated in Steps 1 and 2 above.
3) Prepare reaction mixture as described in the previous paragraph (add in the order specified):
   2^{nd} Round Reaction Mixture:

| Z | μl | MilliQ H₂O |
|---|---|---|
| 25 | μl | 4X QUIC buffer |
| 10 | μl | sample volume from 1^{st} round reaction |
| Y | μl | rPrP^{C} |
| 100 | μl | total volume |

Wherein Z = 73 − Y

Note: Vortex first three components for 5 s prior to adding the rPrP^{C}. Add the rPrP^{C} gently, to avoid creating bubbles. Cap reaction tubes, but do not vortex. Proceed as with steps 4-7 of 1^{st} QuIC round.
4. Digest 10 μl of sample with 3 μg/ml Proteinase K for 1 hour at 37° C. in 1% Sarkosyl/PBS
5. Add 15 μl of 2× Sample Buffer containing 4 M urea to each sample
6. Vortex samples for 1 minute
7. Place in boiling water bath for 10 minutes
8. To eliminate beads, place samples on magnet for 2 minutes and transfer supernatant to fresh tubes
9. Load samples onto SDS-PAGE gel (15 μl/well) & analyze by Western Blot (see Orru et al., 2009)

### D) Real Time (RT)-QuIC Reaction

### Materials:

a. Reaction plates: 96 well Optical Bottom Plate
b. Hamster (90-231) rPrP^{C} or Ha-S full length rPrP^{C} in 10 mM Sodium Phosphate Buffer (pH5.8)
c. Real-Time QuIC Buffer (RTQB), [10 mM phosphate buffer (pH 7.4), 300-400 mM NaCl, 0.1 mg/mL rPrP^{C}, 10 μM Thioflavin T (ThT), and 10 mM ethylenediaminetetraacetic acid tetrasodium salt (EDTA)]
d. 0.05% SDS/PBS for 15B3 beads pre-treatment
e. Freshly made: 0.032 g ThT/10 ml in MilliQ H₂O (10 mM)

### Protocol 1^{st} Round Reaction:

1. Following immunoprecipitation as described in section B, mix 15B3 beads stored in 1×PBS in a 1:1 ratio with 0.05% SDS/PBS and vortex
2. Incubate 15B3 beads at room temperature for at least 15 min, vortexing every ˜5 min
3. Prepare RTQB cocktail (considering 96 μl of cocktail per well×number of wells to be seeded+2 extra wells)
4. RT-QuIC mixture per well:

| MilliQ water | X | μl |
|---|---|---|
| 5X PBS Buffer: | 20 | μl |
| 2 M NaCl: | 8.5 or 13.5 | μl |
| 100 mM EDTA: | 1 | μl |
| 10 mM ThT | 1 | μl |
| rPrP^{C} | Y | μl |
| Seed (15B3 beads + 0.05% SDS/PBS) | 4 | ul |

Each reaction in each well has a final volume of 100 μl. “Y” is the volume of rPrPC that is added to each reaction to have a final concentration of 0.1 mg/ml. This volume varies depending on the concentration of the protein added. Thus the volume of MilliQ water added per reaction (“X”)-varies depending on the volume of rPrP^{C} added to that specific reaction.
   Vortex first five components for 5 s prior to adding the rPrP^{C}, gently invert after adding rPrP^{C}
5. Aliquot 96 μl of RTQ cocktail per well
6. Seed RT-QuIC reaction with 4 μl of 15B3 beads+0.05% SDS/PBS directly in well
7. Seal the plate with plate sealer (Nalge Nunc International 265301)
8. Incubate the plate in BMG Polarstar plate reader at 42-46° C., and measure ThT fluorescence every ˜15 minutes with a shaking kinetic cycle.
9. Shaking program: 1 minute shaking at 700 rpm Double Orbital, then 1 minute resting, except for the final 1 minute for fluorescence measurement.
10. Fluorescence measurement settings:
   excitation: 450 nm, emission: 480 nm
   bottom read, number of flashes: 20
   manual gain: 1000, Integration time: 20 μs

### Substrate Replenishment Step:

11. Make 10 mM ThT stock in MilliQ H₂O (weigh 0.032 g ThT/10 ml MilliQ H2O, filter, and keep on ice)
12. Prepare RT-QuIC cocktails as follows:
13. RT-QuIC mixture per well:

| MilliQ water | X | μl |
|---|---|---|
| 5X PBS Buffer: | 20 | μl |
| 2 M NaCl: | 8.5 or 13.5 | μl |
| 100 mM EDTA: | 1 | μl |
| 10 mM ThT | 1 | μl |
| rPrP^{C} | Y | μl |
| Seed (15B3 beads + 0.05% SDS/PBS) | 0 | μl |

14. Spin plate @ 3000×g for 10 min
15. Take off & discard plate sealer
16. Pipette off 90 μl from each well taking care NOT to touch the bottom of the well with pipette tip
17. Gently add 100 μl/well of fresh substrate to the same wells
18. Seal plate again (fresh sealer) and run RT-QuIC as detailed in steps 8-10.

### Example 2

### Additional Material and Methods

Recombinant Prion Protein Purification:

Syrian golden hamster (residues 23-231; accession K02234), human (residues 23-231; accession #M13899.1) rPrP^{C} and hamster-sheep chimera rPrP^{C} [Syrian hamster residues 23-137 followed by sheep residues 141-234 of the R₁₅₄,Q₁₇₁ polymorph (accession #AY907689)] were amplified and ligated into the pET41 vector (EMD Biosciences) and sequences were verified. Protein expression and purification were performed as previously described (see, for example, Wilham et al., PLoS. Pathog. 6:e1001217, 2010; Atarashi et al., Nat. Methods 4:645-650, 2007). Purity of rPrP^{C} proteins was ≧99% as estimated by SDS-PAGE, immunoblotting and mass spectrometry (data not shown).

Plasma Sample Collection and Tissue Homogenate Preparation:

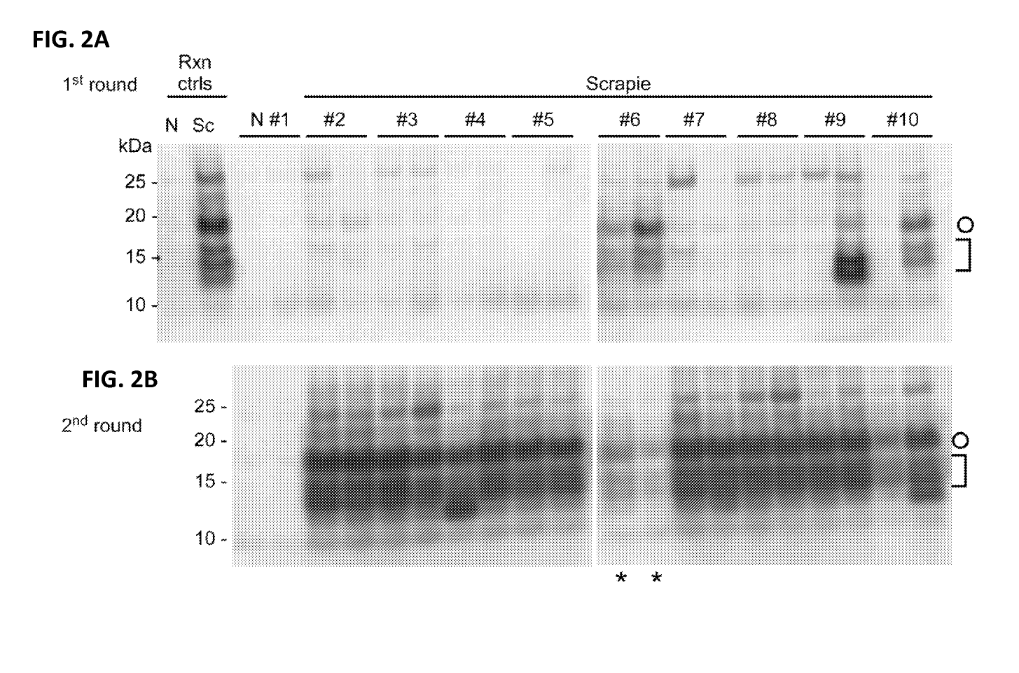

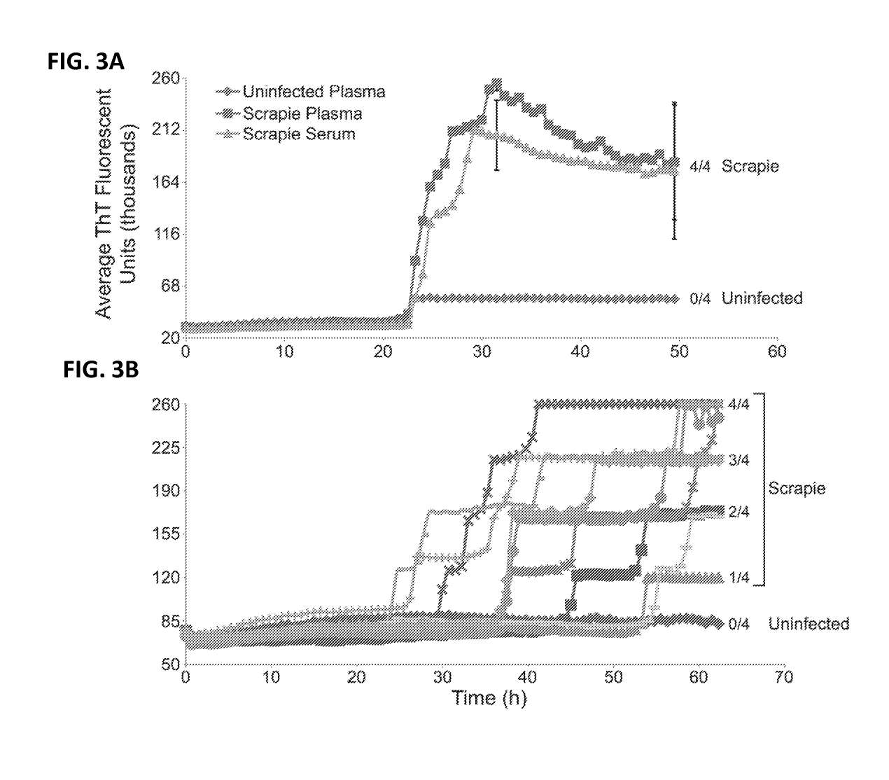

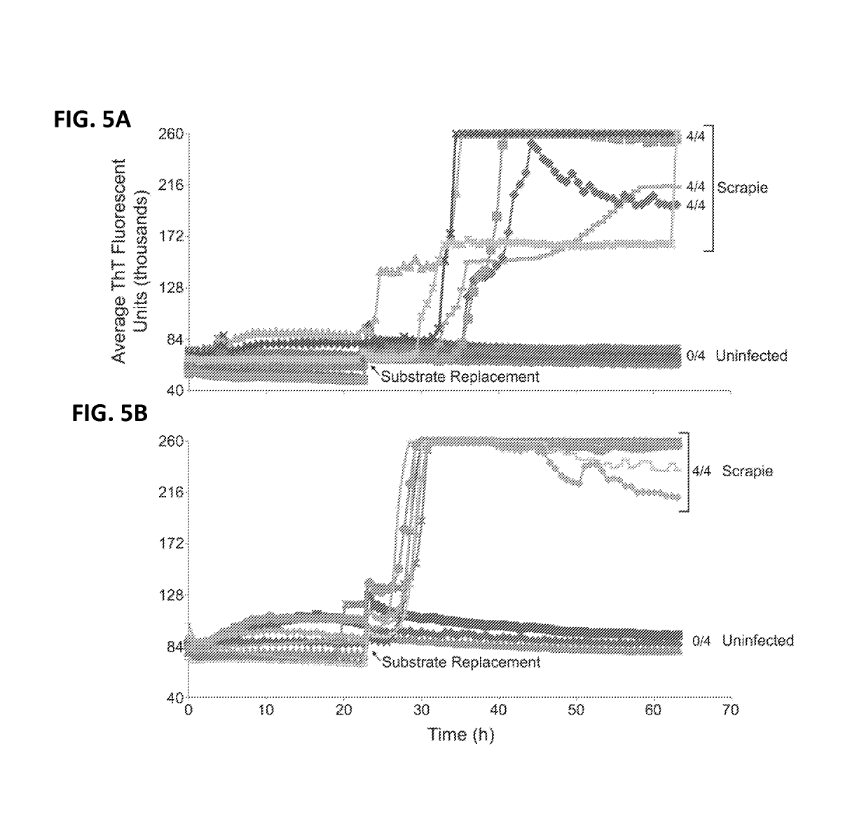

Syrian golden hamsters were inoculated intracerebrally with 50 μl 1% brain homogenate (BH) (FIG. 5) or 10⁸-fold diluted BH (FIGS. 2 and 3) from hamsters clinically affected with the 263K scrapie strain and held for the designated time periods prior to brain tissue or blood collection. The hamsters inoculated with the lower dose of scrapie took longer to become ill so tissues were collected from “near terminal” hamsters at 103-116 dpi for FIGS. 2 & 3 compared to 80 dpi for FIG. 5. For plasma collections, hamsters were euthanized by deep isofluorane anesthesia and exsanguinated via heart stick. Blood was immediately transferred to BD Vacutainer (sodium citrate, Becton-Dickinson) tube and mixed gently. Samples were centrifuged at 3000 rpm in a Beckman J6-HC centrifuge for 15 minutes (min). Plasma was transferred to a new tube and stored at −80° C. Hamster serum samples were collected in a similar fashion but with no sodium citrate. When designated (i.e., FIG. 2B), plasma samples were centrifuged at 16 k relative centrifugal force (rcf) for 30 seconds (s) after thawing and immediately prior to the immunoprecipitation step (using the plasma supernatant). Pooled human plasma (Innovative Research) was stored at −20° C. For brain BH dilution spiking experiments, human plasma aliquots were thawed over night at 4° C. and subjected to a 10 min 2000×g spin to eliminate precipitated fraction.

Hamster and human 10% (w/v) BH were made as previously reported (Saa et al., J. Biol. Chem. 281:35245-35252, 2006), aliquoted and stored at −80° C. For spiking experiments, BH was serially diluted in either 1% or 0.1% SDS in phosphate buffered saline (PBS) with 130 mM NaCl and N2 media supplement (Gibco) (20,21,24), for S-QuIC or RT-QuIC assays, respectively. Two microliters of the designated BH dilutions were used to spike 0.5 ml of human plasma.

15B3 Coating of Magnetic Beads:

Rat anti-Mouse IgM Dynabeads (Invitrogen) were vortexed for 30 seconds and 250 μl of beads (1×10⁸ total beads) were transferred to a new tube for the coating procedure. Following incubation on the magnet for 2 minutes, bead storage buffer was discarded and two washes with 5 original suspended bead volumes using Coating Buffer (0.1% BSA in PBS; made fresh, filtered and kept at 4° C.) were performed. A ratio of 1×10⁶ beads per μg of 15B3 antibody (Prionics) was used. Tubes were incubated with “end-over-end” rotation at room temperature for 2 h. Next, three more washes with Coating Buffer were carried out and beads were resuspended in Coating Buffer (initial bead volume) and stored at 4° C. Mock control beads were prepared as described for 15B3 beads but with no addition of 15B3 antibody.

Preparation of MAGNABIND™ Beads:

MAGNABIND™ heads (Pierce, Rockford, Ill.) were vortexed for 30 s and 1.6×10⁷ total beads were transferred to a new tube. The beads were rinsed twice with 500 μl of 0.5% Triton X-100 in PBS and resuspended in their initial volume with Assay Buffer (TBS, 1% Triton X-100, 1% Tween 20).

Immunoprecipitation of 263K and vCJD PrP-res in Plasma:

15B3 coated beads, mock beads or MAGNABIND™ beads were briefly vortexed and 1.6×10⁷ total beads were transferred to a new tube. Following a 2 minute (min) incubation on the magnet, the storage (coating) buffer was discarded and 500 μl of Immunoprecipitation Buffer (Prionics) was added. Next, 500 μl of BH-spiked human plasma or 500 μl of hamster plasma from uninfected or scrapie-positive animals was added to the beads. Samples were incubated with “end-over-end” rotation at room temperature or 37° C. over night (ON). Subsequently, samples were incubated on the magnet for 2 min, plasma-buffer mix was discarded, and beads were washed twice with 500 μl of Wash Buffer (Prionics). All beads were resuspended into 10 μl of PBS and used fresh.

S-QuIC and RT-QuIC:

The S-QuIC assay was performed as previously described (Atarashi et al., Nat. Methods 5:211-212, 2008; Orrú et al., Protein Eng Des Sel 22:515-521, 2009). 15B3 coated- or mock bead S-QuIC reactions were each seeded with 2 μl of beads in phosphate buffered saline (PBS). The RT-QuIC was performed as previously described (Wilham et al., PLoS. Pathog. 6:e1001217, 2010) except for a few modifications. Briefly, 15B3 coated-, mock or MagnaBind beads from the immunoprecipitation step (resuspended in 10 μl PBS) were combined with 0.05% SDS/PBS (1:1 ratio), incubated at room temperature for 20 minutes and reactions were seeded with 4 μl of 0.05% SDS/PBS-bead mix. RT-QuIC reactions were incubated at 46° C. unless indicated otherwise in figure legends. Substrate replacement was performed by interrupting the RT-QuIC reaction after 24 h, and spinning the plate at 3000×g for 10 minutes at 4° C. Next, 90 μl of supernatant were removed from each well, taking care not to perturb the beads, and 100 μl of new reaction buffer containing fresh rPrP^{C} was gently added to each well. RT-QuIC was continued for an additional 36-60 h.

### Example 3

### Immunoaffinity Capture of Prions from Blood Plasma for SQ Assays

Attempts were made to detect prions spiked into human and sheep plasma samples by directly adding plasma aliquots to the SQ and RTQ assays. However, plasma components strongly inhibited both assays, consistent with previously reported inhibition of another related assay (Trieschmann et al., 2005). These inhibitors might be serum lipoproteins that are known to bind prions (Safar et al., 2006). Accordingly methods were devised to capture and concentrate prions in a detectable form from plasma.

Prion immunoaffinity beads were prepared by coupling monoclonal antibody 15B3 to magnetic beads. This antibody has a strong preference for binding PrP-res and other PrP oligomers over PrP^{C} (Korth et al. Nature 390:74-77, 1997; Biasini et al., J. Neurochem. 105:2190-2204, 2008; Biasini et al., PLoS. ONE. 4:e7816, 2009). The ability of 15B3-coupled beads to capture 263K hamster brain PrP^{Sc} from 0.5 ml human plasma was first tested using the SQ assay. As described previously (Atarashi et al., Nat. Methods 5:211-212, 2007; Atarashi et al., Nat. Methods 4:645-650, 2008), positive reactions were indicated by the characteristic pattern of 17-, 13-, 12-, and 11-kD protease-resistant rPrP-res^{(Sc)} bands in immunoblots. Initial experiments indicated more sensitive detection of sheep PrP-res using the Ha-S substrate along with 0.1% SDS pre-treatment.

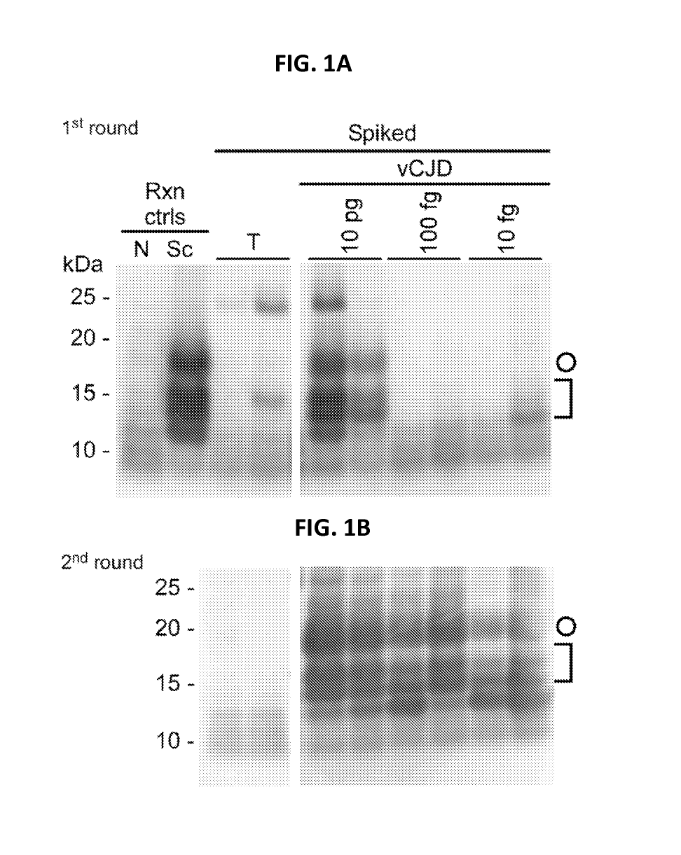

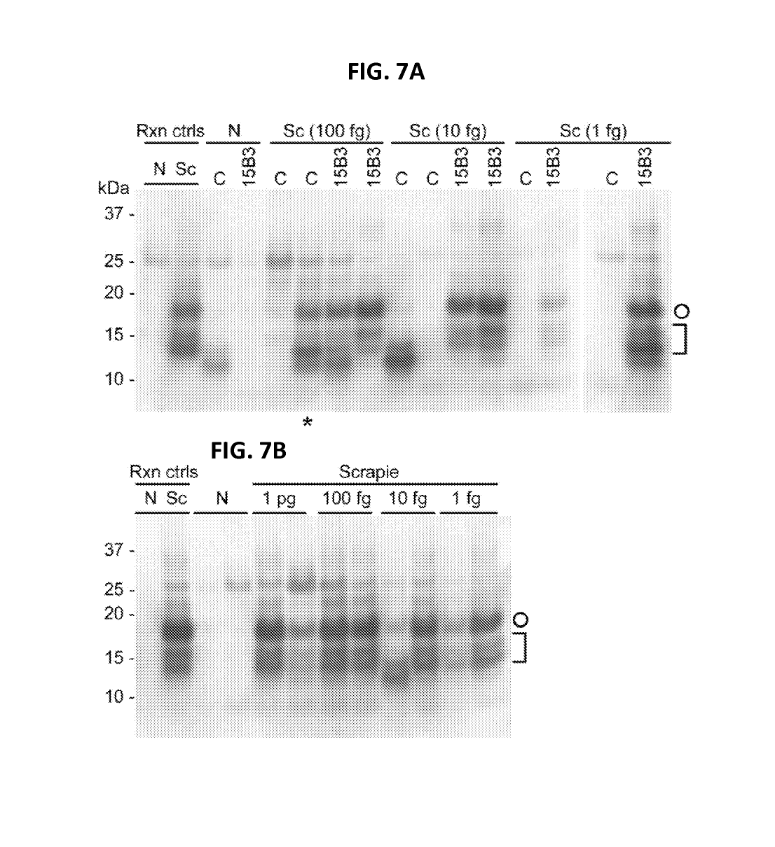

Two-round reactions were performed by seeding aliquots of first-round reaction products into fresh rPrP^{C} substrate. Control (mock) beads coated only with anti-IgM antibodies (without 15B3) had some affinity for prions, as indicated, for example, by the positive rPrP-res^{(Sc)} products generated in one of the two replicate single-round reactions seeded with beads incubated with plasma spiked with a 4×10⁻⁹ dilution of scrapie brain homogenate containing ˜100 fg PrP-res (FIG. 7, lane marked by the asterisk). However, 15B3-coated beads were ˜100-fold more efficient at capturing lower levels of prions from plasma, enabling detection of dilutions containing ≧1 fg PrP-res (FIGS. 7a and 7b). This IP-S-QuIC protocol gave positive reactions from as little as 4×10⁻¹⁰ dilutions of vCJD brain homogenate containing ˜10 fg of human PrP-res (FIG. 1) and 2×10⁻¹¹ dilutions of scrapie hamster brain containing ˜1 fg of PrP-res (FIG. 7). In contrast, no positive rPrP-res^{(Sc)} reaction products were obtained in reactions seeded with non-TSE human or hamster brain homogenates. Moreover, 15B3 IP-S-QuIC detected prion activity naturally present in 0.5 ml of plasma from nine near-terminal scrapie-infected hamsters while no positive S-QuIC reactions were seeded by plasma from a negative control hamster in 2-round reactions (FIG. 2).

### Example 4

### 15B3 IP of Prions in Plasma for Detection by RT-QuIC (eQuIC)

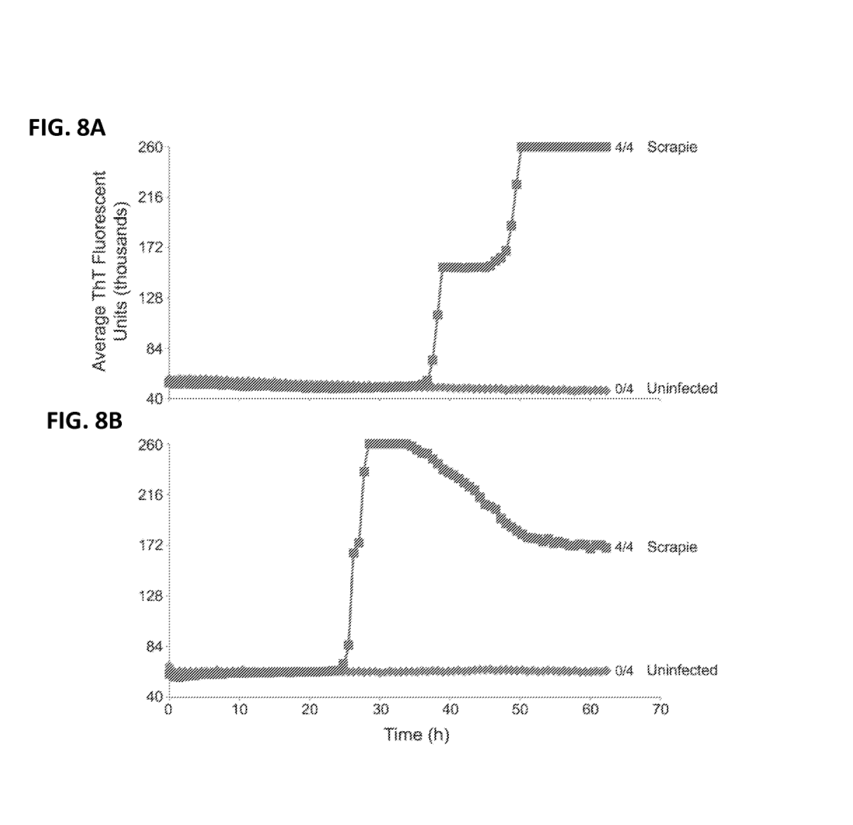

The 15B3 IP was adapted to detection by RT-QuIC (designated IP-RT-QuIC). The RT-QuIC assay uses intermittent shaking of reactions in 96-well plates, rPrP^{C} as the substrate, virtually detergent- (≦0.002% SDS) and chaotrope-free conditions, and ThT-based detection of prion-seeded amyloid fibrils (Wilham et al., supra, 2010; Atarashi et al., Nat. Med. 17:175-178, 2011). Positive reactions are indicated by an enhancement of ThT fluorescence in the presence of rPrP amyloid fibrils, which can be plotted as the average fluorescence from replicate wells. In screening for conditions that allow the detection of prions captured on 15B3 beads from blood plasma with the RT-QuIC assay, it was found that pre-incubation of the prion-bound beads with 0.05% SDS for ˜20 min at room temperature, in addition to a Sarkosyl wash of the beads, accelerated prion amplification in the otherwise detergent-free RT-QuIC (FIG. 8).

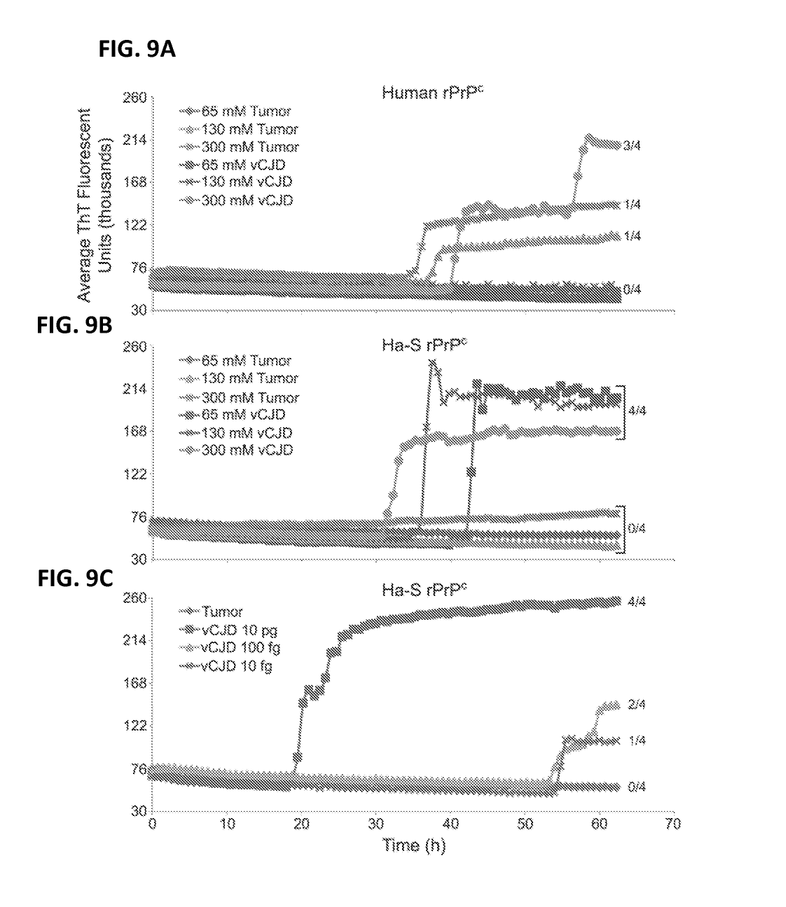

The IP-RT-QuIC protocol detected ˜10⁻¹⁰ dilutions of scrapie brain in human plasma, but was less sensitive for vCJD brain (FIG. 9C). For detecting scrapie, hamster rPrP^{C} 90-231 was used as a substrate. For vCJD, it was found that a chimeric rPrP^{C} molecule, comprised of Syrian hamster residues 23-137 followed by sheep residues 141-234 (R₁₅₄,Q₁₇₁ polymorph), provided for greater sensitivity and less spontaneous (prion-independent) conversion to ThT-positive products than was observed with the homologous human PrP^{C} 23-231 construct (FIG. 9).

Using the hamster rPrP^{C} 90-231 substrate, 15B3 IP of PrP^{Sc} endogenous to 0.5 ml plasma or serum from scrapie-affected hamsters yielded some, but usually not all, positive replicate reactions indicating that the PrP^{Sc} levels in these samples were at, or near, the detection limit (FIG. 3). Collectively, these initial results showed that 15B3 beads captured highly diluted prions from plasma or serum in a manner compatible with both S-QuIC or RT-QuIC detection, but the sensitivity of IP-RT-QuIC was borderline for detecting prions endogenous to scrapie hamster plasma.

### Example 5

### Enhanced QuIC (eQuIC) Detection of 15B3-Captured Prions with Substrate Replacement

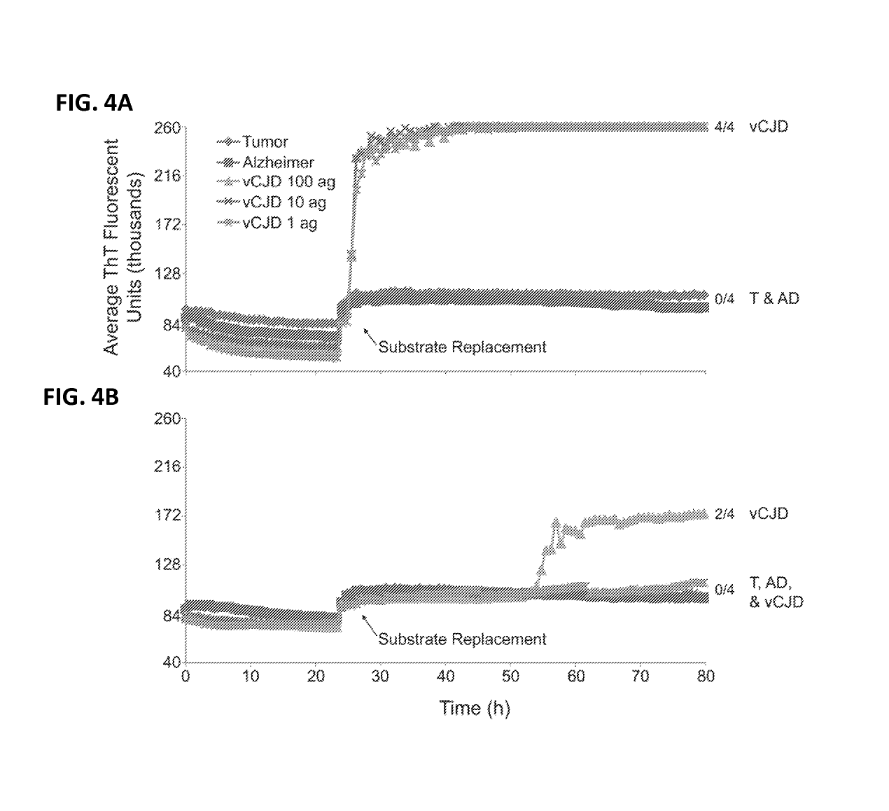

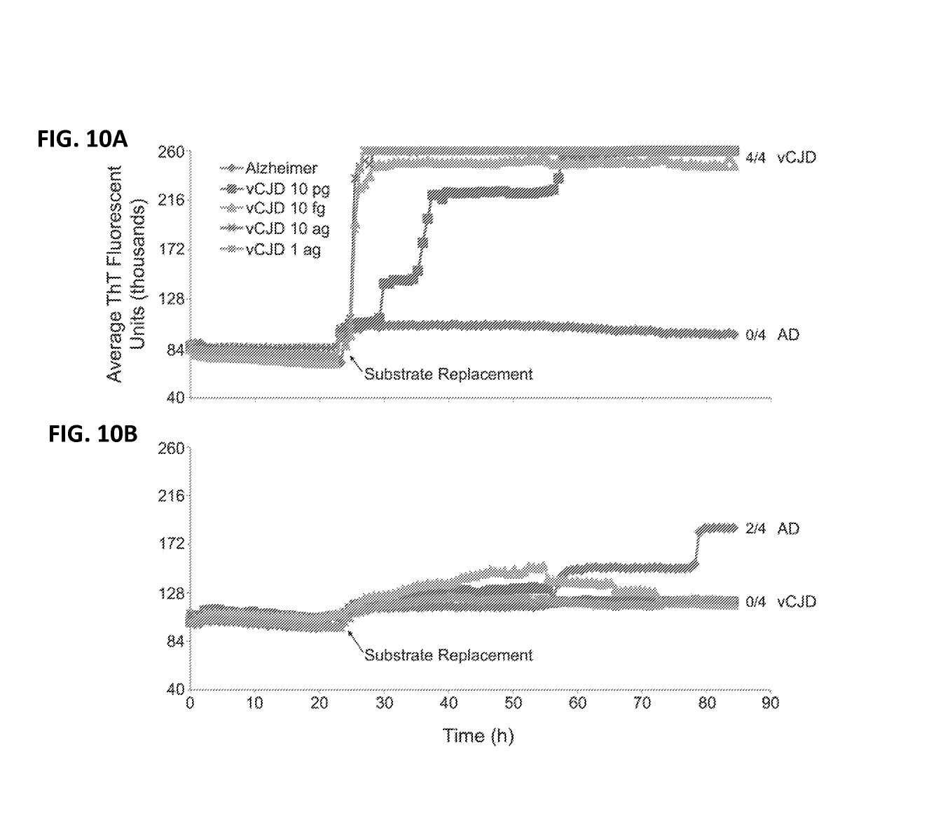

To improve the sensitivity of IP-RT-QuIC a substrate replacement step was introduced after ˜24 h of the RT-QuIC reaction. In IP-RT-QuIC reactions, the beads and associated prions or prion-induced RT-QuIC conversion products tended to adhere to the bottom of reaction wells. Thus, reaction fluid could be removed and fresh rPrP^{C} added while retaining most of the beads or bead-bound reaction products in the well. This combination of IP and RT-QuIC with substrate replacement, which we call enhanced QuIC (eQuIC), allowed detection of 4×10⁻¹⁴ dilutions of vCJD brain tissue (˜1 ag vCJD PrP-res) within ˜28 h in all replicate reactions (n=4) in 3 independent experiments (see, for example, FIG. 4A) performed using four different lots of human plasma. With a further 4×10⁻¹⁵ dilution, 3 of 4 replicate reactions were positive in a single experiment (data not shown). By comparison, Alzheimer's and tumor brain negative control dilutions gave uniformly negative reactions in each of these eQuIC experiments. Mock beads lacking 15B3 gave much reduced sensitivity and consistency (FIG. 4b and FIG. 10). Moreover, the 15B3-coupled beads provided for ≧10⁶-fold more sensitive eQuIC detection than superparamagnetic nanoparticles that were reported recently to have prion-binding capacity (Miller et al., J. Virol. 85:2813-2817, 2011) (FIG. 10). These results showed the ability of the 15B3-based eQuIC to detect extremely low concentrations of prions spiked into human plasma.

### Example 6

### eQuIC Detection of Endogenous Prions in Hamster Plasma Samples

It was also tested if eQuIC improved the detection of prions endogenous to plasma from scrapie-infected hamsters. In contrast to the earlier results with the unenhanced RT-QuIC (FIG. 3), all of the replicate eQuIC reactions from a total of 13 scrapie hamsters were positive, while none of those from 11 uninfected hamsters were positive within 65 h (FIG. 5). Of the scrapie-infected hamsters, 9 were clinically affected (80 days post infection, dpi), and 4 were subclinical (3 at 30 dpi; 1 at 10 dpi). Thus eQuIC detected prions in plasma long before clinical signs of scrapie which, in this model, begin at ˜60 dpi. With some scrapie samples the replicate wells, although all individually positive, gave submaximal average fluorescence values (FIG. 5a). This variability, as well as lag-phase variability, appeared to be due to aggregated plasma components because these variations were not seen with samples that were precleared by brief centrifugation immediately prior to immunoprecipitation (FIG. 5b).

Collectively, the results showed that prions can be captured from a complex inhibitor-laden biological fluid in a manner that is compatible with ultrasensitive detection by in vitro prion amplification assays. The eQuIC assay in particular provided a practical, high-throughput and rapid means of testing for amounts of PrP-res (1 ag) that was several orders of magnitude below those typically required to cause prion disease by intracerebral inoculation into animals. The ability of eQuIC to detect prions in plasma samples raises indicates that this assay can be used to improve prion disease diagnosis in humans and animals, and to screen the blood supply for prion contamination. Discrimination of scrapie-infected and uninfected hamsters based on eQuIC analysis of their blood plasmas samples was demonstrated. As 5-10 fold more CJD infectivity has been found in leukocyte fractions of blood (Brown et al., Haemophilia. 13 Suppl 5:33-40, 2007), eQuIC analysis of leukocytes would be very sensitive.

The two-stage substrate addition disclosed herein for the eQuIC differs from serial (multiple-round) amplification steps that were used in protein misfolding cyclic amplification (PMCA) (Saa et al., Science 313:92-94, 2006; Saa et al., J. Biol. Chem. 281:35245-35252, 2006), rPrP-PMCA (Atarashi et al., Nat. Methods 4:645-650, 2008) and QuIC (Atarashi et al., Nat. Methods 5:211-212, 2008; Atarashi et al, Nat. Methods 4:645-650, 2007) reactions because most of the bead-bound prions and prion-seeded products are retained in the reaction vessel, so that the substrate can be replaced without removing most of the seed particles. In contrast, in serial PMCA and S-QuIC reactions, only a small proportion (typically ≦10%) of the total reaction is transferred to a new vessel containing fresh substrate so that much of the seeding activity from the first round is lost.

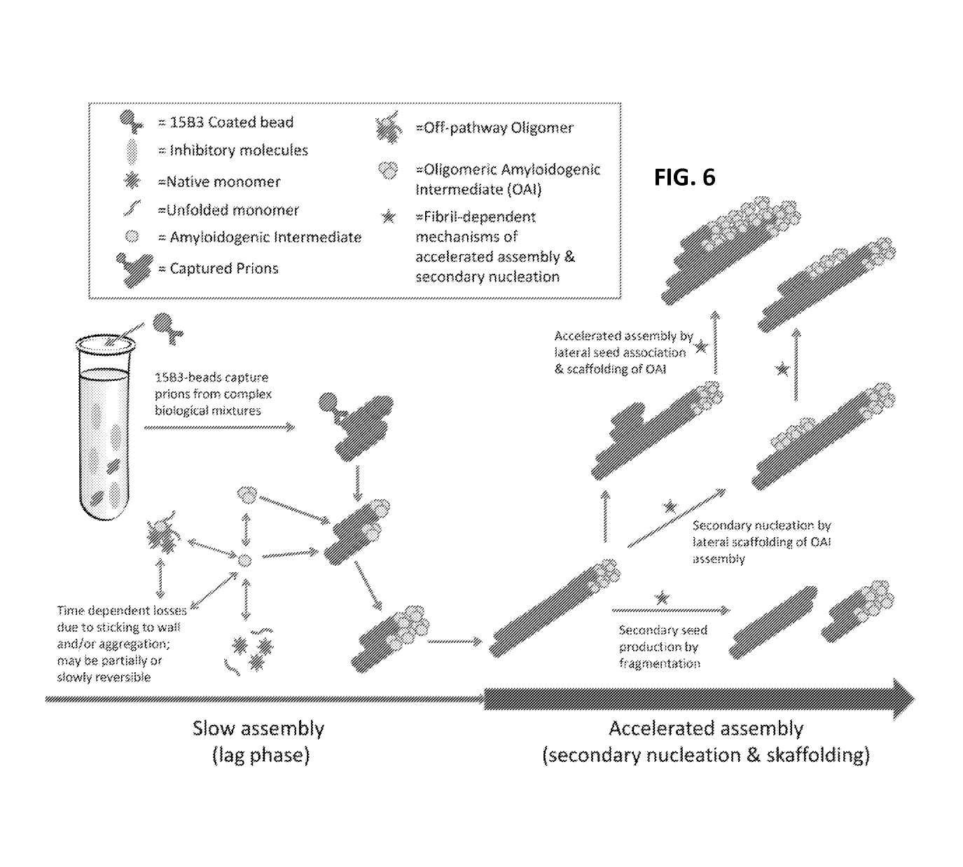

Without being bound by theory, the results suggest that at least two processes are occurring during the initial lag phase of the eQuIC reaction, specifically between the addition of seed and substrate replacement (FIG. 6). First, the rPrP^{C} can be moving into a pool that is less rapidly accessible to prion-seeded fibril assembly, such as an off-pathway oligomer (OO); otherwise, the addition of fresh rPrP^{C} after 20 h, but before the initial substrate is converted to detectable ThT-positive fibrillar products, would not accelerate the reaction. Thus, a “less rapidly accessible” pool rather than an inaccessible pool is suggested, because even without substrate replenishment the vast majority of the substrate can still be converted if given enough time. Secondly, the initial seed must be being altered and primed in some way to seed more rapid fibril assembly upon the addition of fresh substrate; otherwise, it would have been capable of seeding rapid ThT-positive rPrP-res^{(Sc)} assembly at the beginning of the reaction, when there was the same concentration of fresh substrate. Again, without being bound by theory, this priming effect might be explained by secondary-nucleation mechanisms (Ferrone et al., J. Mol. Biol. 183:611-631, 1985; Padrick and Miranker, Biochemistry 41:4694-4703, 2002), such as those marked with red stars in FIG. 6. For example during the lag phase, prion seeds may elongate by incorporating rPrP^{C} at a relatively slow and largely undetected rate determined in part by the concentration of seed particles. With continued elongation, the seeded rPrP fibrils would become long enough to be sheared by agitation, increasing the seed particle concentration and accelerating overall fibril assembly. Moreover, other types of fibril-dependent secondary nucleation might contribute to the acceleration of fibril assembly. For instance, fibril assembly might be hastened by the pre-alignment or scaffolding of rPrP^{C} substrate or amyloidogenic intermediate (AI) along the sides of an existing fibril, either with or without the need for a similarly aligned seed. In any case, further studies will be required to define the mechanistic underpinnings of the effects of 2-phase substrate addition.

The more effective rPrP^{C} substrate for the RT-QuIC is not always one that is most homologous with the type of prion/PrP-res being assayed. Surprisingly, the substrate that worked best for the detection of human vCJD was the chimeric hamster-sheep construct (Ha-S rPrP^{C}), rather than a human rPrP^{C} molecule.

The IP-RT-QuIC assay offers considerable advantages when compared to other ultrasensitive prion/PrP^{Sc} assays. Relative to the first generation RT-QuIC assay, the eQuIC not only allows for prion detection in inhibitor-laden samples such as plasma, but also enhances the sensitivity for vCJD brain homogenate dilutions into human plasma by at least 10.000-fold. Compared to PMCA reactions that have been described, the IP-RT-QuIC is more rapid for a given sensitivity level, more practical by using bacteria rather than brain as the source of PrP^{C} substrate, more easily replicated by using shaking rather than sonication, and more amenable to high-throughput analyses due to multiwell plate-based reactions and fluorescence detection.

Edgeworth (Lancet 377:487-493, 2011) have produced a vCJD PrP-res detection assay which includes prion capture on stainless steel beads and an ELISA detection method. Whereas this capture-ELISA assay detected 10¹⁰-fold dilutions of vCJD brain homogenate in whole blood, the eQuIC assay disclosed herein detected 10¹⁴-fold dilutions in plasma. The Edgeworth assay detected PrP^{vCJD} in blood from 15 symptomatic patients with a ˜70% sensitivity and 100% specificity, which is nearly as effective as the RT-QuIC in diagnosing sporadic CJD using CSF samples (Atarashi et al., Nat. Med. 17:175-178, 2011). The ˜10,000-fold greater sensitivity of the eQuIC assays that are disclosed herein in detecting brain-derived vCJD seeding activity provides improved sensitivity of vCJD and sCJD diagnosis using blood, plasma, CSF or other samples. The assays disclosed herein also have use in a wide variety of materials such as foods, feeds, transplanted tissues, medical devices, agricultural wastes and byproducts, soils, water sources, and other environmental samples.

### Example 7

### 15B3 Capture of Prions for Detection by RT-QuIC

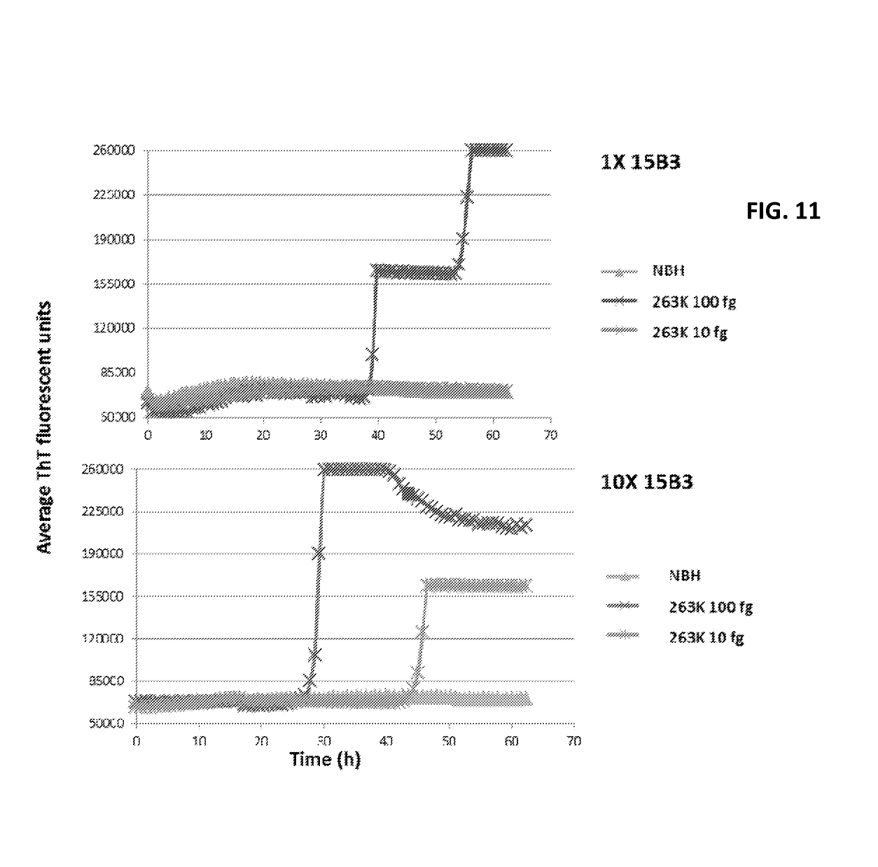

Conditions were sought that allow for the detection of prions captured on 15B3 beads from blood plasma with the RTQ assay. There were additional factors that improved the speed and sensitivity of RTQ detection. Specifically, 1) Coupling of 12-fold higher concentration of 15B3 antibody to the beads (FIG. 11): the higher antibody density on the beads may help compensate for potential PrP-res binding inhibitors in plasma and/or accelerates binding by providing a higher concentration of binding sites; treatment of the prion-bound beads with 0.05% SDS for 15-20 min, at RT, in addition to the Sarkosyl wash prior to RTQ. In some examples, the reactions utilize 0.360 μg of 15B3 per 4×10⁵ Dynabeads.

Initially, it was found that prion seeding activity could be eluted from the beads with 1 M NaCl prior to SDS treatment and addition to RTQ reactions. As noted above, the eluted material required a pre-incubation with 0.05% SDS for 15-20 min, at room temperature, to get enhanced prion specific seeding activity in the RTQ.

In further tests, it was found that a portion of the prion-bound beads (i.e., ⅕ of the total beads resuspended in 10 μl of 1×PBS) obtained from the IP and SDS-treatment steps could more simply be mixed directly with rPrP^{C} substrate in the RTQ reaction plate well to initiate the reaction as described in detail in the protocol in Example 1.

The amount of 15B3 antibody loaded onto the beads was doubled (“20×”) to determine if increased concentration of the antibody on the beads would increase sensitivity of the assay for sheep ARQ brain homogenate (containing 100 fg or 10 pg PrP-res) spiked into 0.5 ml sheep plasma. In these studies “20×” indicates 200 μg 15B3 incubated with 1×10⁸ total beads and “10×” indicates 100 μg 15B3 incubated with 1×10⁸ total beads). Ha-S rPrP^{C} was used as the substrate. Improved sensitivity (see the dilution containing 100 fg PrP-res) was achieved with higher loading of 15B3.

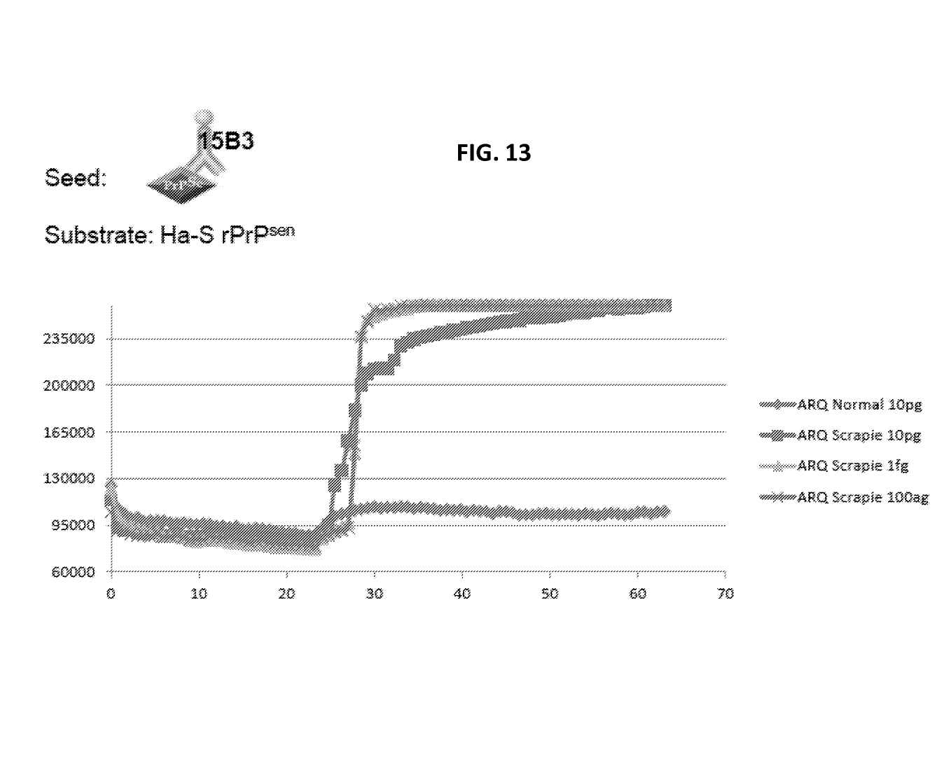

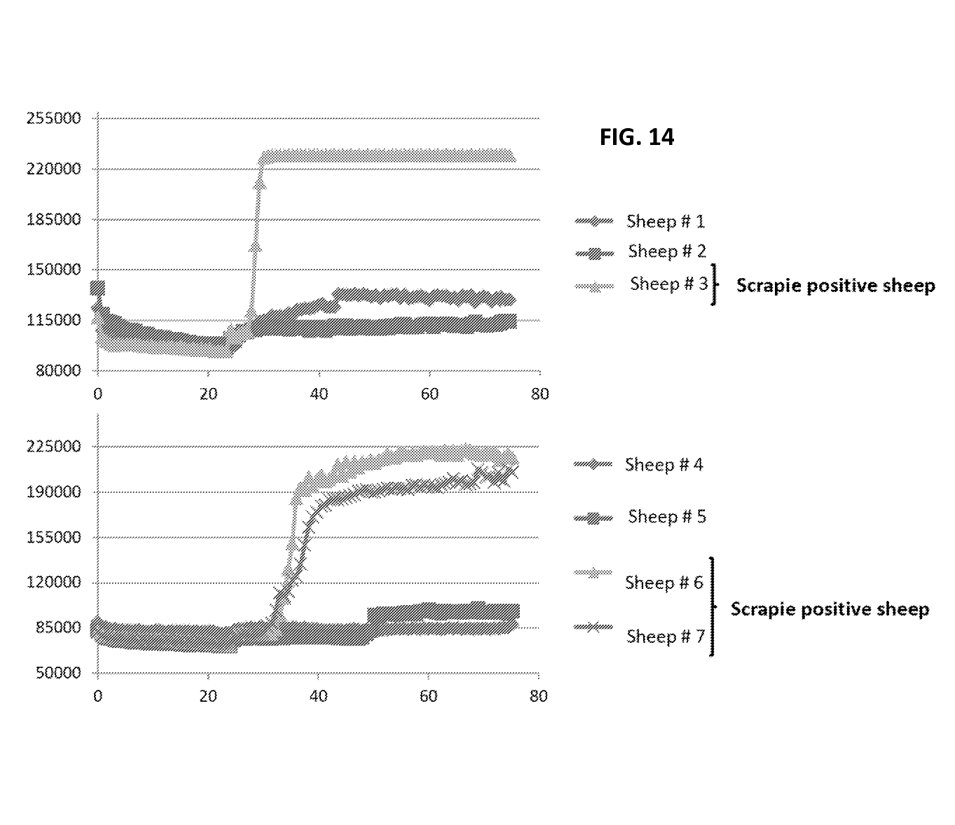

The “20×” eQuIC conditions were used to detect sheep ARQ scrapie brain homogenates (containing down to 100 ag PrP-res) spiked into 0.5 ml plasma (see FIG. 13, four replicate reactions). The same eQuIC condition were used to test whether the assay can detect prion seeding activity endogenous to 0.5 ml plasma samples from scrapie infected sheep (ARQ|VRQ, VRQ\VRQ), as opposed to brain homogenate spiked in plasma. The results (see FIG. 14) showed clear discrimination between 3 scrapie-positive and 4 normal sheep. All replicate reactions (n=4) were positive with each of the prion containing samples and negative for samples that did not contain prions.

### Example 8

### Additional eQuIC Assays

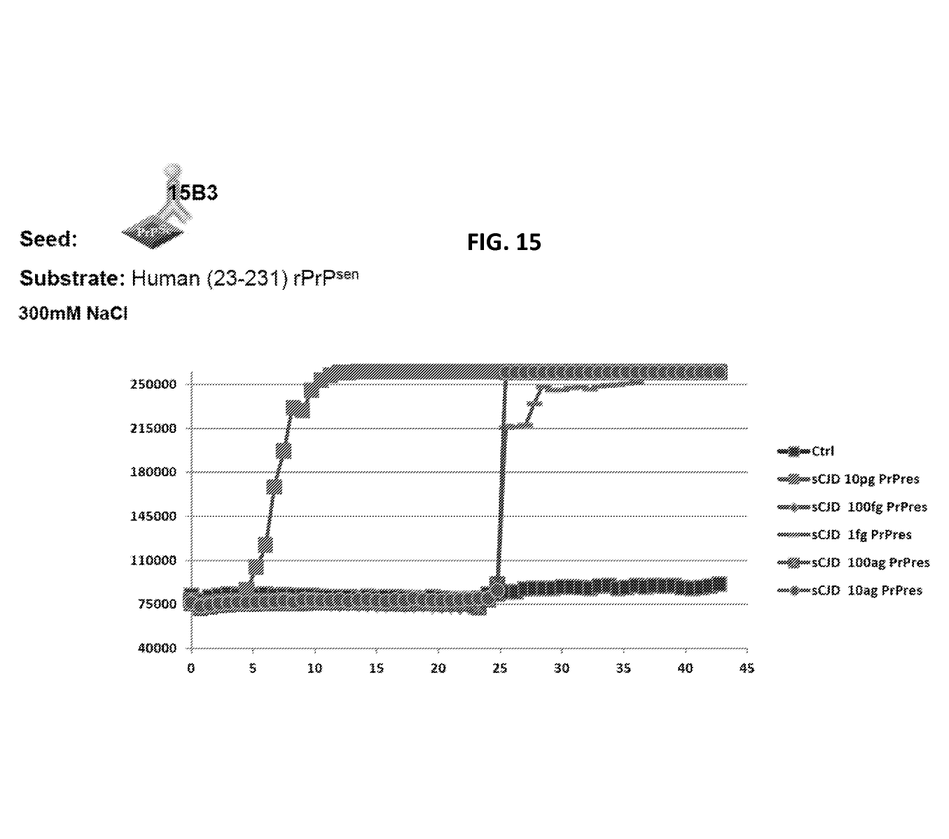

eQuIC was also used to detect sporadic CJD brain homogenate spikes into 0.5 ml normal human plasma. Dilutions of sCJD brain homogenates down to those containing 1 fg PrP-res were detected in all replicate reactions (n=4) in both 300 and 400 mM NaCl. Substitution of the Ha-S rPrP^{C} substrate for the human rPrP^{C} resulted in similar sensitivity for sCJD, specifically down to dilutions containing 1 fg PrP-res. These reactions used full length human rPrP-sen as the substrate. Dilutions containing as little as 10 ag of PrP-res were detected in all replicate reactions (n=4), while those spiked with a 10⁻⁴ dilution of control (National Institute for Biological Standards and Control (NIBSC, UK) NHBZO/0001 normal brain tissue) gave no responses (see FIG. 15).

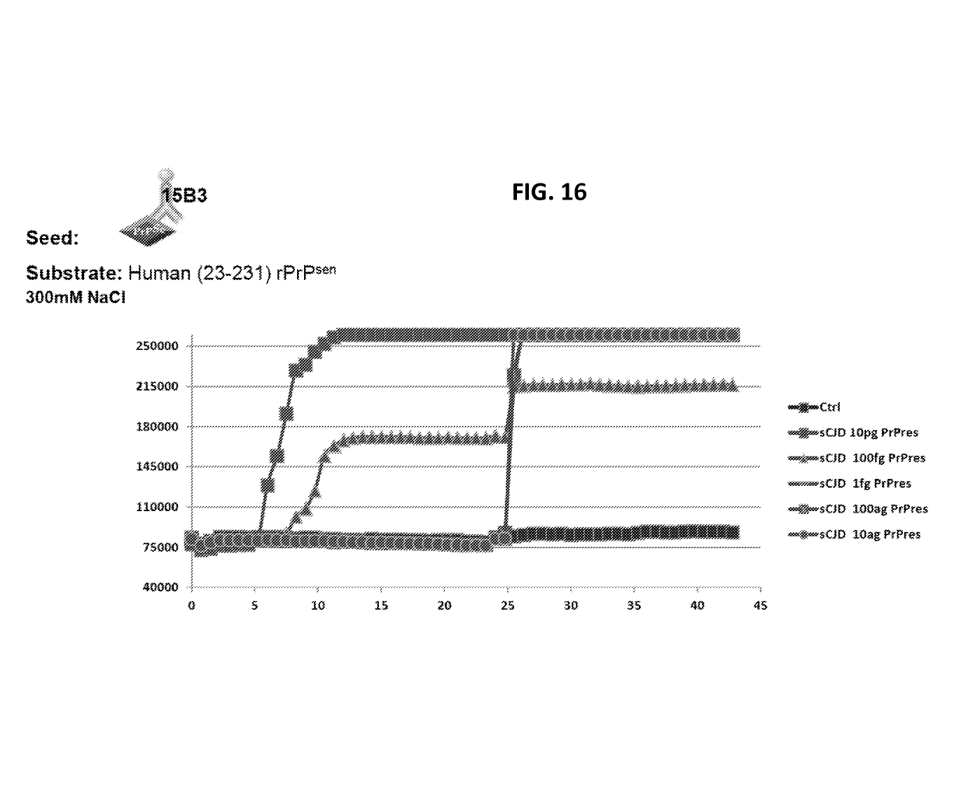

An analogous experiment was done in which sCJD brain homogenate dilutions were spiked into 0.5 ml normal human cerebrospinal fluid. Dilutions containing as little as 10 ag of PrP-res were detected in all replicate reactions (n=4), while those spiked with a 10⁻⁴ dilution of control ((National Institute for Biological Standards and Control (NIBSC, UK) NHBZO/0001 normal brain tissue) gave no responses (see FIG. 16).

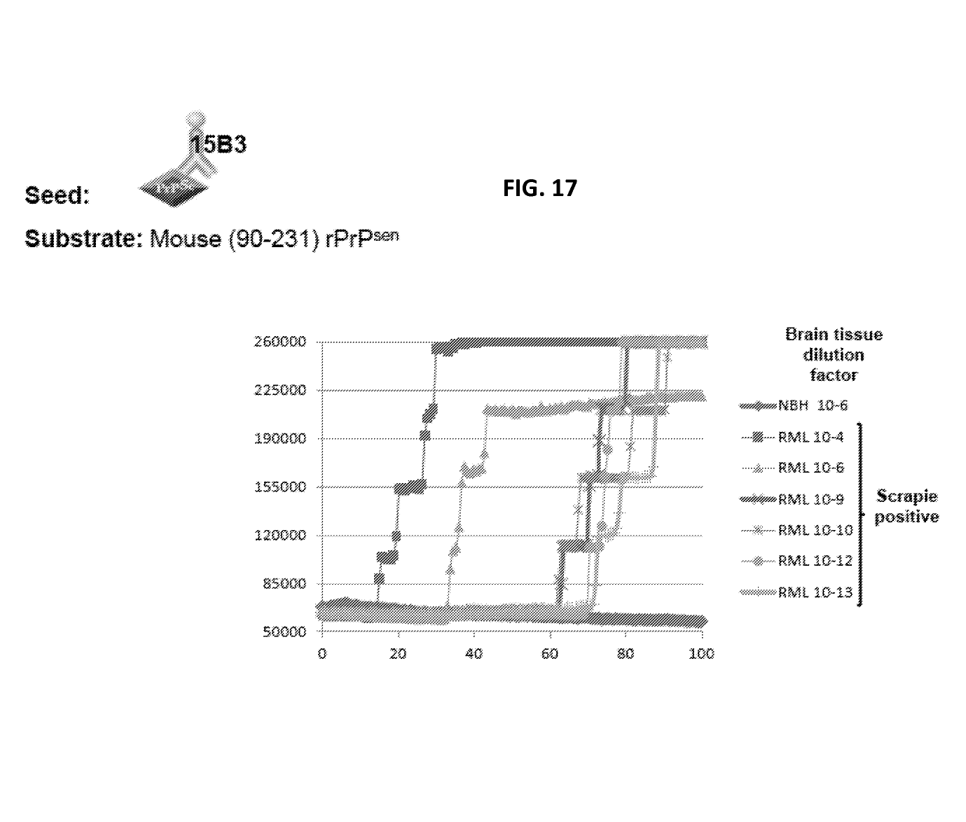

eQuIC detection of mouse RML scrapie brain homogenate dilutions in 0.5 ml of mouse plasma were also performed. These reaction conditions were similar to those described above (46° C., 300 μg of 15B3 with 1×10⁸ total beads) except mouse rPrP^{C} 90-231 was used as a substrate. All replicate reactions (n=4) were positive for dilutions down to 10⁻¹³ (containing 100 ag PrP-res) while those seeded with a 10⁻⁶ dilution of normal mouse brain gave no positive reactions within 100 h (FIG. 17).

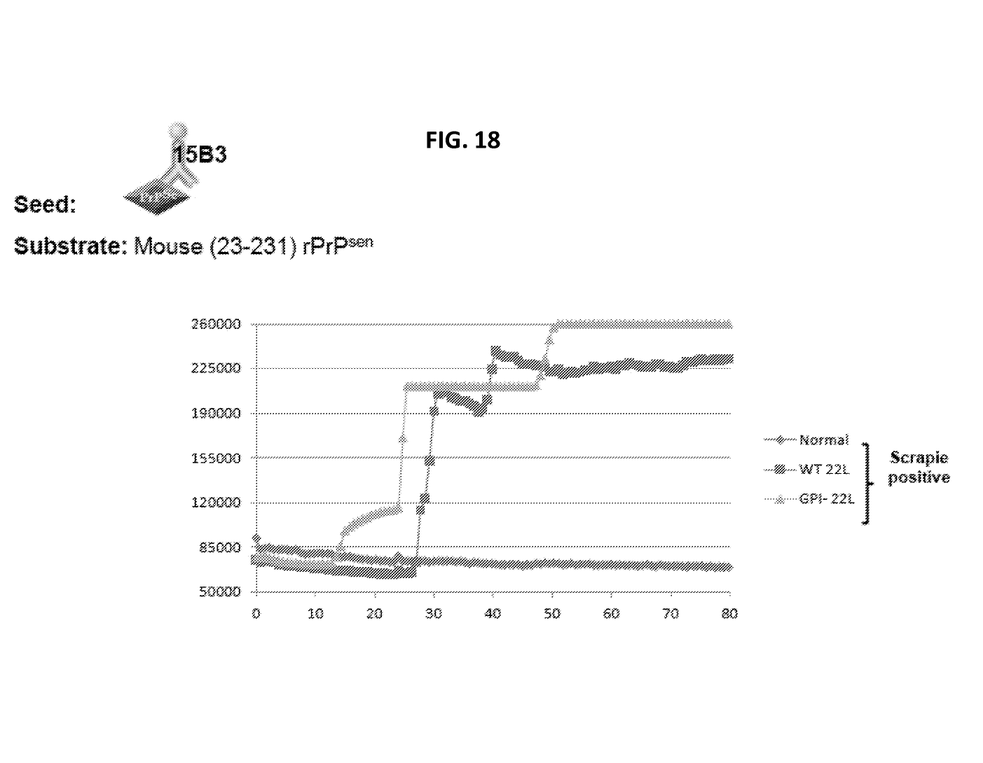

eQuIC detection was also used to identify prion seeding activity endogenous to 200 μl of plasma of 22 L scrapie-infected mice. Single samples were tested, one from a wild-type mouse and another from a transgenic mouse expressing only PrP-sen that lacks the glycophosphatidylinositol anchor. In both cases, all replicate reactions (n=4) gave positive responses, while no positive responses were observed from a plasma sample from an uninfected wild-type mouse (FIG. 18).

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of detecting prion protein, comprising:
contacting a sample with an effective amount of an antibody that specifically binds prions, PrP^{Sc} or PrP-res for sufficient time to form an immune complex;
separating the immune complex from the sample;
mixing the immune complex with purified recombinant prion protein (rPrP^{C}) to make a reaction mixture; and
performing an amplification reaction comprising:
(i) incubating the reaction mixture to permit coaggregation of the PrP-res with the rPrP^{C} that is present in the reaction mixture;
(ii) maintaining incubation conditions that promote coaggregation of the rPrP^{C} with the PrP-res to result in a conversion of the rPrP^{C} to rPrP-res^{(Sc)} while inhibiting development of rPrP-res^{(spon)};
(iii) agitating aggregates formed during step (i), wherein the reaction conditions comprise shaking the reaction mixture without sonication; and
(iv) repeating steps (i)-(iii)
detecting rPrP-res^{(Sc)} in the reaction mixture, wherein detection of rPrP-res^{(Sc)} in the reaction mixture indicates that PrP-res was present in the sample.

2. The method of claim 1, wherein the antibody that specifically binds the prions, PrP-res or PrP^{Sc} is coupled to a solid substrate.

3. The method of claim 2, wherein the solid substrate is a magnetic bead.

4. The method of claim 3, wherein separating the immune complex comprises the use of a magnet.

5. The method of claim 1, wherein the antibody is 15B3, a humanized form thereof or an antigen binding fragment thereof.

6. The method of claim 5, wherein the antibody is coupled to a magnetic bead, and wherein the concentration of the antibody on the magnetic beads is about 360 μg/ml, or wherein the concentration of the antibody on the magnetic beads is about 10-500 μg of 15B3 per 1×10⁸ number of beads.

7. The method of claim 1, wherein the sample is contacted with the antibody at a temperature of about 19-40° C.

8. The method of claim 1, wherein the sample is a biological sample.

9. The method of claim 8, wherein the biological sample is a blood, plasma, serum or cerebrospinal fluid sample.

10. The method of claim 3, comprising incubating the magnetic beads with a buffer comprising sodium dodecyl sulfate or Sarkosyl following contacting the biological sample with the magnetic beads.

11. The method of claim 10, comprising washing the magnetic beads with 0.01% to 0.1% sodium dodecyl sulfate.

12. The method of claim 10, comprising washing the magnetic beads with about 0.05% sodium dodecyl sulfate.

13. The method of claim 1, wherein detecting the presence of rPrP-res^{(Sc)} comprises the use of thioflavin T (ThT).

14. The method of claim 13, wherein detergent of greater than 0.002% is not included in the reaction mixture.

15. The method of claim 1, further comprising adding additional rPrP^{C} to the reaction mixture without removing rPrP-res^{(Sc)} prior to detecting the presence of rPrP-res^{(Sc)}.

16. The method of claim 15, wherein the additional rPrP^{C} is added to the reaction mixture without serial rounds of amplification.

17. The method of claim 1, wherein the rPrP^{C} is a chimeric hamster-sheep rPrP^{C}, and wherein the PrP-res is PrP^{CJD}.

18. The method of claim 17, wherein the hamster-sheep rPrP^{C} comprises amino acids 23-137 of the Syrian hamster PrP sequence and residues 141-234 of sheep PrP.

19. Them method of claim 18, wherein the sheep PrP comprises R154 and Q171.

20. The method of claim 1, wherein performing the amplification reaction comprises incubating the reaction mixture in 0.05% to 0.8% of a detergent.

21. The method of claim 20, wherein the detergent comprises sodium dodecyl sulfate.

22. The method of claim 21, wherein the detergent comprises 0.05-0.4% sodium dodecyl sulfate (SDS) and 0.05-0.4% Triton X-100.

23. The method of claim 20, wherein the detergent comprises 0.4% sodium dodecyl sulfate (SDS) and 0.4% Triton X-100.

24. The method of claim 1, wherein detecting the presence of PrP-res comprises contacting the reaction mixture with a second antibody that specifically binds prions, PrP-res, or PrP^{Sc}.

25. The method of claim 24, wherein the second antibody that specifically binds PrP-res is not 15B3.

26. The method of claim 20, wherein detecting the presence of PrP-res comprises an enzyme linked immunosorbant assay (ELISA), a radioimmunoassay (RIA), a lateral flow assay, a Surround optical fiber immunoassay (SOPHIA) or a Western blot.

27. The method of claim 1, further comprising quantitation the PrP^{Sc}.

28. The method of claim 1, wherein agitating the aggregates comprises shaking the reaction mixture without sonication for a period of time that is substantially equal to a period of rest that precedes the shaking.

29. The method of claim 28, wherein the reaction mixture is shaken for about 60 seconds and then not shaken for about 60 seconds.

30. The method of claim 1, wherein step (iii) is repeated from about 1 to about 200 times.

31. A method of detecting prion protein, comprising:
contacting a biological sample with an effective amount of antibody 15B3 coupled to a solid substrate for sufficient time to form an immune complex on the solid substrate;
separating the immune complex on the substrate from the biological sample;
washing the immune complex on the solid substrate with a buffer comprising 0.5% sodium dodecyl sulfate.
mixing the immune complex on the solid substrate with purified hamster sheep chimeric recombinant prion protein (rPrP^{C}) and Thioflavin T to make a reaction mixture; and
performing an amplification reaction comprising:
(i) incubating the reaction mixture to permit coaggregation of the PrP-res with the rPrP^{C} that is present in the reaction mixture;
(ii) maintaining incubation conditions that promote coaggregation of the rPrP^{C} with the PrP-res to result in a conversion of the rPrP^{C} to rPrP-res^{(Sc)} while inhibiting development of rPrP-res^{(spon)};
(iii) agitating aggregates formed during step (i), wherein the reaction mixture is shaken for about 60 seconds and then not shaken for about 60 seconds;
(iv) adding additional hamster sheep chimeric recombinant prion protein (rPrP^{C}) to the reaction mixture prior to the formation a detectable rPrP-res^{(Sc)}; and
(v) optionally repeating step (iii); and/or
(vi) detecting the rPrP-res^{(Sc)} in the reaction mixture using fluorescence, wherein fluorescence of the reaction mixture indicates that PrP-res was present in the sample.

32. The method of claim 31, wherein the biological sample is a blood, serum, plasma, cerebral spinal fluid or tissue sample from a human.


SEQUENCE LISTING (12 sequences)

SEQ ID NO: 1 - protein, 209 aa, Mesocricetus auratus
KKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRS

SEQ ID NO: 2 - protein, 208 aa, Mus musculus
KKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRS

SEQ ID NO: 3 - protein, 209 aa, Homo sapiens
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSS

SEQ ID NO: 4 - protein, 209 aa, Homo sapiens
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSS

SEQ ID NO: 5 - protein, 218 aa, Bos taurus
KKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGAS

SEQ ID NO: 6 - protein, 210 aa, Ovis aries
KKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGAS

SEQ ID NO: 7 - protein, 210 aa, Cervus unicolor
KKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGAS

SEQ ID NO: 8 - protein, 254 aa, Mesocricetus auratus
MANLSYWLLALFVAMWTDVGLCKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRSSAVLFSSPPVILLISFLIFLMVG

SEQ ID NO: 9 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 10 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 11 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 12 - protein, 211 aa, Artificial Sequence
  Chimeric hamster-sheep proteinase K-sensitive prion protein
HMKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGAS